(19) United States
(12) Patent Application Publication
(10) Pub. No.: US 2025/0114372 A1
(43) Pub. Date: Apr. 10, 2025

(54) COMPOSITION COMPRISING PHOTOSENSITZER COMPOUND AND ACTIVATION THEREOF

(71) Applicant: Raymond J DIFALCO, Congers, NY (US)

(72) Inventor: Raymond J DIFALCO, Congers, NY (US)

(21) Appl. No.: 18/293,752

(22) PCT Filed: Aug. 4, 2022
(86) PCT No.: PCT/US2022/039099

§ 371 Date: Jan. 30, 2024

Related U.S. Application Data
(60) Provisional application No. 63/250,668, filed on Sep. 30, 2021.
(60) Provisional application No. 63/227,785, filed on Jul. 30, 2021.

(51) Int. Cl. A61K 31/5415; A61K 9/00; A61K 9/1277; A61K 41/00; A61K 47/02; A61N 5/06

(52) U.S. Cl. CPC A61K 31/5415; A61K 9/0056; A61K 9/1277; A61K 41/008; A61K 47/02; A61N 5/062; A61N 2005/0626; A61N 2005/0645; A61N 2005/0652; A61N 2005/0659; A61N 2005/0663; A61N 2005/0664

(57) ABSTRACT

Photosensitizer compositions effective against various microbial infections, in addition to methods for making the same, are disclosed. In addition, phototherapy devices and methods for delivering light to tissues to activate photoactive agents are also disclosed. Further, methods for using the phototherapy device in combination with photosensitizer compositions are described.

### RELATED APPLICATIONS

This application is a U.S. National Stage application of International Patent Application Number PCT/US22/39099, filed on Aug. 2, 2022, which claims benefit of priority to provisional application No. 63/227,785, filed on Jul. 30, 2021, and provisional application No. 63/250,668, filed on Sep. 30, 2021, the disclosures of all of which are incorporated herein by reference in their entirety.

### TECHNICAL FIELD

This disclosure relates to photosensitizer compositions for prevention and/or treatment of various microbial infections, enveloped viruses, and/or associated conditions. This disclosure also relates to methods for preparing the photosensitizer compositions, devices comprising the compositions, and methods for using the composition and the devices in conjunction with each other for preventing and/or treating various antimicrobial infections and/or associated conditions.

### BACKGROUND

Photodynamic therapy (PDT) (also known as phototherapy or light therapy) includes a photosensitizer (PS) capable of photo activation by absorption of visible light to form reactive oxygen species (ROS). PCT can be employed in a wide range of therapeutic diseases and/or cosmetic conditions. Phototherapy by either employing LED or laser as light source has been used to treat viral infections (including, but not limited to, HIV, HHV-6, Hepatitis A, B, C, Covid-19), wounds, injuries, neck pain, osteoarthritis, the side effects of chemotherapy and radiotherapy, for instance, in addition to lime disease and acne.

Phenothiazine and derivatives thereof are an established class of photosensitizers that are amphipathic planar aromatic molecules having phototoxic efficiency against a broad range of microorganisms. Derivatives and analogs of phenothiazine such as Methylene blue (MB), 1-Octanol and 1,3-diphenylisobenzofuran (DPIBF), Rose Bengal (RB), new methylene blue (NMB) and dimethyl methylene blue (DMMB) Azure A (AA), azure C (AC), azure B (AB), toluidine blue O (TBO), brilliant crystal blue (BCB), pyronin Y (PYY) and neutral red (NR) have been recognized as clinically important molecules.

It is an object of the present disclosure to provide compositions comprising phenothiazine derived compounds for use in treating and/or preventing microbial infections and/or associated conditions. It is further an object of the present disclosure to provide devices, comprising phenothiazine compounds, for use in phototherapy in conjunction with either pharmaceutical or nutraceutical ingredients.

### SUMMARY

Disclosed herein is a composition comprising a phenothiazine derived compound. In one example, a composition disclosed herein is a pharmaceutical composition comprising a therapeutically effective amount of the phenothiazine derived compound. In one example, the phenothiazine derived compound is methylene blue.

In one example, a composition disclosed herein comprises a phospholipid selected from the group consisting of phosphatidylcholine and/or lyso-phophatidylcholine.

In another example, a composition disclosed herein comprises one or more metal nano particles selected from the group consisting of a nano or micronized gold, nano or micronized silver, nano or micronized zinc, nano or micronized copper, nano or micronized iridium, nano or micronized platinum, nano or micronized silica, and nano or micronized palladium.

In yet another example, a composition disclosed herein comprises one or more metal nano particles selected from the group consisting of a nano or micronized colloidal gold, nano or micronized colloidal silver, nano or micronized colloidal zinc, nano or micronized colloidal copper, nano or micronized colloidal iridium, nano or micronized colloidal platinum, nano or micronized colloidal silica, and nano or micronized colloidal palladium.

In yet another example, a composition disclosed herein comprises polyethylene glycol (PEG).

In yet another example, a composition disclosed herein comprises polysorbate forms.

In yet another example, a composition disclosed herein comprises glycerin.

In yet another example, a composition disclosed herein comprises purified water.

In yet another example, a composition disclosed herein comprises one or more ingredients selected from the group consisting of mannitol, corn starch, fish gelatin, bovine gelatin, mint flavor, sucralose, zanthem gum, Carbopol 934, gaur gum, and a suitable edible gelling agent.

In one example, a composition disclosed herein is in a form selected from the group consisting of solid, liquid or solution, lotion, cream, ointment, gel, paste, and aerosol foam or spray. In another example, a composition disclosed herein is in a form selected from an oral solution, a tablet, a capsule, and a lozenge.

In one example, a composition disclosed herein can be selected from the group consisting of oral composition, topical composition, intranasal composition, and intravenous composition.

In one example, disclosed herein is a method for preparing the composition.

In one example, disclosed herein is a device for use in phototherapy or photodynamic therapy. In one example, the device comprises at least 1 LED light to at least 10 LED lights having a wavelength ranging from about 600 nm to about 750 nm.

In one example, disclosed herein is a method for treating a condition in a subject comprising administering or applying a composition as described above to a subject in need thereof.

In one example, disclosed herein is a method for treating a condition in a subject comprising administering or applying the composition to a subject in need thereof and exposing tissue or skin of the subject to a device described herein, wherein the device is placed 1/16″ to 4″ from the skin surface.

In one example, disclosed herein is a device for treating a condition in a subject comprising exposing tissue or skin of a patient in need thereof to the device.

In one example, disclosed herein is a kit comprising the composition.

In one example, disclosed herein is a kit comprising the device.

These and other objects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

The embodiments disclosed herein are illustrated in the accompanying drawings. The embodiments herein will be better understood from the following description with reference to the drawings, in which:

FIG. 1 is an illustration of the cellular mechanism of action of photosensitizer.

FIG. 2 are graphs depicting fluorescence intensity and rate of fluorescence of MB conjugated with gold nanoparticles.

FIG. 3 depicts a graph showing increment in fluorescence intensity by Phenothiazinium category molecule.

FIG. 4 are images from TEM, SEM, and Confocal Laser Scanning Microscopy (CLSM) analysis showing photo-cytotoxicity by phenothiazinium category molecule.

FIG. 5 depicts TEM image of Methylene blue-liposomes and a graph showing Dynamic light scattering (DLS) measurement of MB-liposomes.

FIG. 6 is UV-Vis's spectra of free Methylene Blue (MB) and MB-mixture, and a graph depicting stability of composition-hydrodynamic diameter of MB-liposomes over 14 days.

FIG. 7 are TEM images of Phenothiazinium category molecule and a graph showing nano particle size.

FIG. 8 are images depicting liposome entrapped Phenothiazinium category molecule, Phenothiazinium category molecule-liposome after removal of coating, and enzymatic treatment of Phenothiazinium category molecule-liposome; and a graph depicting particle size distribution before and after treatment; and zeta potential of various Phenothiazinium category molecule.

FIG. 9 depicts graph showing 24-h cell viability (by MTT assay) of free MB and MB-liposomes and Distilled Water (DI) as control at different concentrations with dark treatment and photodynamic therapy (PDT) treatment, and the images depict free MB and MB-liposomes at 8 μm concentration and Distilled Water (DI) with dark treatment and photodynamic therapy (PDT) treatment.

FIG. 10 are SEM images of Curcumin nanoparticles and silver and gold nanoparticles.

FIG. 11 are SEM images of colloidal gold nanorods.

FIG. 12 are images depicting Phenothiazinium category molecule, Phenothiazinium category molecule liposomal form, oxidation rate, and Phenothiazinium category molecule liposomal form after sonification.

FIG. 13 is a pictorial representation of blue light excitation of Liposomal-MB and red-light excitation of Liposomal-MB respectively.

FIG. 14 is a graph depicting X ray diffraction pattern of Colloidal silver nanoparticles.

FIG. 15 is a graph showing X ray diffraction pattern of pure curcumin.

FIG. 16 depicts ESI mass spectrum of methylene blue before and after overnight irradiation respectively.

FIG. 17 is a graph showing nano silver mass spectrometry pattern.

FIG. 18 depicts Mass spectra Pattern of Curcumin.

FIG. 18(a) represents mass spectra of curcumin

FIG. 18(b) represents mass spectra of hydroxylated curcumin.

FIG. 18(c) is mass spectra of daughter ion of curcumin.

FIG. 19 are graphs showing absorption spectra of colloidal gold ion of 5 nm and 20 nm and colloidal gold with low molecular complex respectively.

FIG. 20 is a schematic overview of photo inactivation of multidrug resistant microbes by monomeric methylene blue conjugated gold nanoparticles.

FIG. 21 is a schematic representation depicting tailoring photosensitive ROS for advance photodynamic therapy; and Jablonski diagram showing the photo chemical mechanism.

FIG. 22 is a schematic representation of Tissue penetration depths of various wavelengths.

FIG. 23 is a graph with the central image shows photo spectrometric data measured from penetration through a human hand in vivo; and

FIG. 24 is a schematic representation of general action mechanism of photodynamic applications on viruses.

FIG. 25 depicts absorption of light in the various layers of the skin (Samson et al. Evidence Report/Technology Assessment 2004, 111, pages 1-97).

FIG. 26 depicts the propagation of light of different wavelengths in the tissues.

FIG. 27 depicts an example of the device, wristband, disclosed herein.

FIG. 28 depicts the wristband comprising additional components.

FIG. 29 depicts a different view of the wristband, disclosed herein.

FIG. 30 depicts yet another view of the wristband, disclosed herein.

FIG. 31 depicts yet another view of the wristband, disclosed herein.

FIG. 32 depicts yet another view of the wristband, disclosed herein.

FIG. 33 depicts the top view of a wristband of the invention.

FIG. 34 depicts the side view of a wristband of the invention.

FIG. 35 depicts another side view of a wristband of the invention.

FIG. 36 depicts a wristband holding container from outside.

FIG. 37 depicts a wristband laid and held in location in such a way that when a liquid bottle, containing a photosensitive drug and/or a composition comprising a photosensitive drug, is placed into the container the height of the wristband, when activated, is in such a way the light emitting from the wrist band hits the side of the bottling holding the liquid.

FIG. 38 depicts an example of a container described herein.

FIG. 39 depicts the backside of a model of the wristband as described herein.

FIG. 40 depicts the sideview of a model of the wristband described herein.

FIG. 41 depicts the top view of the wristband described herein, wherein the wristband is rested on its side.

FIG. 42 depicts the size of the wristband described herein with a side-by-side comparison of the wristband with a hand watch.

FIG. 43 depicts the wristband as described herein, wherein the wristband is illuminated.

FIG. 44 depicts a subject wearing the wristband as described herein.

FIG. 45 depicts the bottom view of the housing component of the wristband, wherein the LED lights in the housing component as illuminated.

FIG. 46 depicts the wristband as described herein rested on its side, wherein the wristband is illuminated.

FIG. 47 depicts the inside of a cap as described herein.

FIG. 48 depicts the outside of a cap as described herein.

FIG. 49A depicts examples of compositions (compositions 1 to 3) as described herein. FIG. 49B depicts examples of compositions (compositions 4 to 6) as described herein. FIG. 49C depicts examples of compositions (compositions 7 to 9) as described herein. FIG. 49D depicts examples of compositions (compositions 10 to 12) as described herein. FIG. 49E depicts examples of compositions (compositions 13 to 15) as described herein. FIG. 49F depicts examples of compositions (compositions 16 to 18) as described herein. FIG. 49G depicts examples of compositions (compositions 19 to 21) as described herein. FIG. 49H depicts examples of compositions (compositions 22 to 24) as described herein. FIG. 49I depicts examples of compositions (compositions 25 to 27) as described herein. FIG. 49J depicts examples of compositions (compositions 28 to 30) as described herein. FIG. 49K depicts examples of compositions (compositions 31 to 33) as described herein. FIG. 49L depicts examples of compositions (compositions 34 to 36) as described herein. FIG. 49M depicts examples of compositions (compositions 37 to 38) as described herein.

### DETAILED DESCRIPTION

The examples herein and the various features and advantageous details thereof are explained more fully with reference to the non-limiting embodiments that are illustrated in the accompanying drawings and detailed in the following description. Descriptions of well-known features and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Unless otherwise indicated, the definitions and embodiments described in this and other sections are intended to be applicable to all embodiments and aspects of the present application herein described for which they are suitable as would be understood by a person skilled in the art.

The singular forms “a”, “an” and “the” as used herein include plural references unless the content clearly dictates otherwise. For example, an embodiment including “an agent” should be understood to present certain aspects with one compound or two or more additional compounds.

The term “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) as used herein are inclusive or open-ended and do not exclude additional, unrecited elements or process steps.

The term “consisting of” and its derivatives as used herein are intended to be close ended terms that specify the presence of stated features, elements, components, groups, integers, and/or steps, and also exclude the presence of other unstated features, elements, components, groups, integers and/or steps.

The term “consisting essentially of, as used herein, is intended to specify the presence of the stated features, elements, components, groups, integers, and/or steps as well as those that do not materially affect the basic and novel characteristic(s) of these features, elements, components, groups, integers, and/or steps.

The term “about” is used herein to mean a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

The term “and/or” as used herein means that the listed items are present, or used, individually or in combination. In effect, this term means that “at least one of or “one or more” of the listed items is used or present.

The term “suitable” as used herein means that the selection of the compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material is administered to an individual without causing significant undesirable biological effects or significantly interacting in a deleterious manner with any of the components of the composition in which it is contained.

The term “nutraceutical” as used herein denotes usefulness in both the nutritional and pharmaceutical field of application. Thus, the novel nutraceutical compositions can find use as supplement to food and beverages, dietary supplement and as pharmaceutical compositions for enteral or parenteral application which may be solid compositions such as capsules or tablets, or liquid compositions, such as solutions or suspensions. As will be evident from the foregoing, the term nutraceutical composition also comprises food and beverages containing the ingredients described herein.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient. “Treat,” “treating” and “treatment” can also mean prolonging survival as compared to expected survival if not receiving treatment. “Treat,” “treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

The terms “administer,” “administering”, “administration,” and the like, as used herein, refer to the methods that are used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that in some instances are employed with the agents and methods described herein include, e.g., as discussed in Goodman and Gilman, The Pharmacological Basis of Therapeutics (current edition), Pergamon; and Remington's, Pharmaceutical Sciences (current edition), Mack Publishing Co., Easton, Pa. In certain embodiments, the agents and compositions described herein are administered orally. In some embodiments, the compositions described herein are administered parenterally.

The term “topical composition” as used herein includes a composition that is suitable for topical application to the skin, nail, mucosa, wound bed or wound cavity. A topical composition may, for example, be used to confer a therapeutic or cosmetic benefit to its user. Specific topical compositions can be used for local, regional, or transdermal application of substances.

The term “topical administration” is used herein to include the delivery of a substance, such as a therapeutically active agent, to the skin or a localized region of the body.

The term “transdermal” as used herein includes a process that occurs through the skin. The terms “transdermal,” “percutaneous” and “transcutaneous” can be used interchangeably. In certain embodiments, “transdermal” also includes epicutaneous. Transdermal administration is often applied where systemic delivery of an active is desired, although it may also be useful for delivering an active to tissues underlying the skin with minimal systemic absorption.

The term “transdermal application” as used herein includes administration through the skin. Transdermal application can be used for systemic delivery of an active agent; however, it is also useful for delivery of an active agent to tissues underlying the skin with minimal systemic absorption. In certain embodiments, “transdermal application” can also include epicutaneous application.

The terms “subject”, “patient” or “individual” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The term “pharmaceutically acceptable salt” means an acid addition salt or basic addition salt which is suitable for or compatible with the treatment of subjects, including human subjects. The term “wt %” means a percentage expressed in terms of weight of the ingredient or agent over the total weight of the composition multiplied by 100.

The term “w/w” as used herein refers to the number of grams of solute in 100 g of solution.

The term “water” as used herein as an ingredient in the compositions of the application refers to pharmaceutically acceptable water.

The term “nano” as used herein refers to particles having a particle size ranging from about 1 nanometer to 100 nanometers.

The term “micronized” as used herein refers to particles having a particle size ranging from about 100 nanometers to about 500 nanometers.

The term “nano or micronized colloidal” as used herein refers to nano sized particles or micronized sized particles suspended in a solution or a liquid or suspension than being added to a solution or suspension, or a gel or tablet.

### Composition

Disclosed herein are compositions comprising liposome structures encapsulating one or more photosensitizers. Liposome, according to embodiments herein, refer to its generally known meaning which includes closed structures comprising one or more outer lipid layer surrounding an internal aqueous space. In an embodiment, the composition is a liposomal composition.

In one example, the composition(s) disclosed herein comprise at least one photosensitizer, at least one phospholipid, at least one excipient, at least one solvent, and at least one carrier. In another example, the composition(s) disclosed herein comprise at least one photosensitizer, at least one phospholipid, at least one metal nanoparticle, at least one excipient, at least one solvent and at least one carrier.

### Photosensitizer

The term “photosensitizer”, as used herein refer to any compound capable of photoactivation, particularly for generating cytotoxic species such as reactive oxygen species (ROS). It includes photosensitizing agents. It further includes salts, or prodrug, or analogs, or derivatives of such photosensitizers. Various photosensitizers are generally known and may be used in various embodiments herein. Examples of photosensitizers include, but is not limited to, prophyrins; dyes such as phenothiazinium, phenodiazinium, phenooxaziniums; chlorophyll; chlorins; bacteriochlorins; phenothiaziniums; prophycenes; and other natural occurring photosensitizing agents, or mixtures thereof. In some embodiments the photosensitizer may be functional using molecules such as functional groups, ligands, etc. The composition may include one or more photosensitizers. In one example, the photosensitizer is a phenothizine compound. In another example, the photosensitizer is a naturally occurring compound such as diferuloylmethane.

### Phenothiazinium

In one example, the composition includes phenothiazinium. According to examples described herein, phenothiaziniums or phenothiazinium compounds, used interchangeably, refer to phenothiazine or phenothiazine derived compounds including, but not limited to, their salts, analogs, prodrugs, conjugates, and/or derivatives. Examples of phenothiazine compounds include, but are not limited to, methylene blue, rose bengal, toluidine blue O, brilliant crystal blue, neutral red, pyronin Y, new methylene blue, dimethyl methylene blue, dimethyl methylene blue, Azure A, Azure B and Azure C. In an example, the composition includes methylene blue. In another example, the composition includes rose bengal. In yet another example, the composition includes toluidine blue. In yet another example, the composition includes brilliant crystal blue. In another embodiment, the composition includes neutral red. In yet another example, the composition includes pyronin Y. In yet another example, the composition includes new methylene blue. In yet another example, the composition includes dimethyl methylene blue. In yet another example, the composition includes Azure A. In yet another example, the composition includes Azure B. Further in an embodiment, the composition includes Azure C. In a preferred example, the phenothiazine compound is methylene blue.

In one example, the composition described herein comprises a phenothiazinium compound (for example, methylene blue) in an amount ranging from 0.5 mg equivalent to 15.0 mg equivalent. In another example, the composition comprises a phenothiazinium compound (for example, methylene blue) in an amount selected from the group consisting of 0.5 mg equivalent, 1.0 mg equivalent, 1.5 mg equivalent, 2.0 mg equivalent, 2.5 mg equivalent, 3.0 mg equivalent, 3.5 mg equivalent, 4.0 mg equivalent, 4.5 mg equivalent, 5.0 mg equivalent, 5.5 mg equivalent, 6.0 mg equivalent, 6.5 mg equivalent, 7.0 mg equivalent, 7.5 mg equivalent, 8.0 mg equivalent, 8.5 mg equivalent, 9.0 mg equivalent, 9.5 mg equivalent, 10.0 mg equivalent, 10.5 mg equivalent, 11.0 mg equivalent, 11.5 mg equivalent, 12.0 mg equivalent, 12.5 mg equivalent, 13.0 mg equivalent, 13.5 mg equivalent, 14.0 mg equivalent, 14.5 mg equivalent, or 15.0 mg equivalent. In some examples, the phenothiazinium compound is present in an amount within a range of the amounts described in this paragraph.

In one example, the composition comprises a phenothiazinium compound (for example, methylene blue) in an amount ranging from 0.01% (w/w) to 15% of the composition. In another example, the composition comprises a phenothiazinium compound (for example, methylene blue) in an amount selected from the group consisting of 0.01% of total weight of the composition, 0.02% of total weight of the composition, 0.03% of total weight of the composition, 0.04% of total weight of the composition, 0.05% of total weight of the composition, 0.06% of total weight of the composition, 0.07% of total weight of the composition, 0.08% of total weight of the composition, 0.09% of total weight of the composition, 0.1% of total weight of the composition, 0.25% of total weight of the composition, 0.5% of total weight of the composition, 0.75% of total weight of the composition, 1.0% of total weight of the composition, 1.25% of total weight of the composition, 1.5% of total weight of the composition, 1.75% of total weight of the composition, 2.0% of total weight of the composition, 2.25% of total weight of the composition, 2.5% of total weight of the composition, 2.75% of total weight of the composition, 3.0% of total weight of the composition, 3.25% of total weight of the composition, 3.5% of total weight of the composition, 3.75% of total weight of the composition, 4.0% of total weight of the composition, 4.25% of total weight of the composition, 4.5% of total weight of the composition, 4.75% of total weight of the composition, 5.0% of total weight of the composition, 5.25% of total weight of the composition, 5.5% of total weight of the composition, 5.75% of total weight of the composition, 6.0% of total weight of the composition, 6.25% of total weight of the composition, 6.5% of total weight of the composition, 6.75% of total weight of the composition, 7.0% of total weight of the composition, 7.25% of total weight of the composition, 7.5% of total weight of the composition, 7.75% of total weight of the composition, 8.0% of total weight of the composition, 8.25% of total weight of the composition, 8.5% of total weight of the composition, 8.75% of total weight of the composition, 9.0% of total weight of the composition, 9.25% of total weight of the composition, 9.5% of total weight of the composition, 9.75% of total weight of the composition, 10.0% of total weight of the composition, 10.25% of total weight of the composition, 10.5% of total weight of the composition, 10.75% of total weight of the composition, 11.0% of total weight of the composition, 11.25% of total weight of the composition, 11.50% of total weight of the composition, 11.75% of total weight of the composition, 12.0% of total weight of the composition, 12.25% of total weight of the composition, 12.50% of total weight of the composition, 12.75% of total weight of the composition, 13.0% of total weight of the composition, 13.25% of total weight of the composition, 13.50% of total weight of the composition, 13.75% of total weight of the composition, 14.0% of total weight of the composition, 14.25% of total weight of the composition, 14.50% of total weight of the composition, 14.75% of total weight of the composition, and 15.0% of total weight of the composition. In some examples, the phenothiazinium compound is present in an amount within a range of the amounts described in this paragraph.

### Curcuminoid

In one example, the composition further includes curcuminoid. The term “curcuminoid”, as used herein, refers to biologically active compounds in the plant Curcuma longa. It includes curcumin and compounds non-curcumin such as desmethoxycurcumin, bisdesmethoxycurcumin, etc. It also includes derivative, conjugates, analogs, etc. of curcuminoids. In one example, the composition includes curcumin, also referred to herein as Diferuloylmethane. The curcuminoids may be included in any suitable form for eg: as extracts such as aqueous, alco-aqueous or alcoholic extract; isolates; or chemically synthesized compounds., Diferuloylmethane is an extract of turmeric (Curcuma Longa plant) in the form of viscous oily fluids, pasty semisolids or hard amorphous solids, and which comprise in varying concentrations, both curcuminoids and essential volatile oil, wherein the volatile oil comprises a mixture of non-curcuminoids in varying concentrations.

In one example, the composition comprises curcuminoid in an amount ranging from 0.5 mg equivalent to 15.0 mg equivalent. In another example, the composition comprises diferuloylmethane in an amount ranging from 0.1% (w/w) to 15% (w/w) of the total weight of the composition. In some examples, the composition comprises Diferuloylmethane in an amount selected from the group consisting of 0.5 mg equivalent, 1.0 mg equivalent, 1.5 mg equivalent, 2.0 mg equivalent, 2.5 mg equivalent, 3.0 mg equivalent, 3.5 mg equivalent, 4.0 mg equivalent, 4.5 mg equivalent, 5.0 mg equivalent, 5.5 mg equivalent, 6.0 mg equivalent, 6.5 mg equivalent, 7.0 mg equivalent, 7.5 mg equivalent, 8.0 mg equivalent, 8.5 mg equivalent, 9.0 mg equivalent, 9.5 mg equivalent, 10.0 mg equivalent, 10.5 mg equivalent, 11.0 mg equivalent, 11.5 mg equivalent, 12.0 mg equivalent, 12.5 mg equivalent, 13.0 mg equivalent, 13.5 mg equivalent, 14.0 mg equivalent, 14.5 mg equivalent, and 15.0 mg equivalent. In some examples, the curcuminoid is present in an amount within a range of the amounts described in this paragraph.

### Phospholipids

In one example, a composition described herein comprises a phospholipid selected from phosphatidylcholine or a lyso-phosphatidylcholine. The phosphatidylcholine can be selected from the group consisting of a hydrogenated phosphatidylcholine, a non-hydrogenated phosphatidylcholine, a hydrogenated sunflower phosphatidylcholine, a non-hydrogenated sunflower phosphatidylcholine, a hydrogenated soybean phosphatidylcholine, a non-hydrogenated soybean phosphatidylcholine, a egg yolk phosphatidylcholine, and a hydrogenated egg yolk phosphatidylcholine. In another example, a composition described herein comprises one or more phospholipids selected from the group consisting of a lysolipid, a lyso-phospholipid, and a conjugate or a derivative of a phospholipid.

In one example, the phosphatidylcholine used in the composition contains a minimum specification of 75% to 100% as detected by HPLC-RI method. In another example, the lyso-phosphatidylcholine used in the composition contains a minimum specification of 0.5 to 10%, as detected by HPLC-PLH method.

In one example, a composition described herein comprises a phospholipid in an amount ranging from 0.1% of total weight of the composition to 50% of total weight of the composition. In another example, a composition described herein comprises a phospholipid in an amount selected from the group consisting of 0.1% of the total weight of the composition, 0.5% of the total weight of the composition, 1.0% of the total weight of the composition, 1.5% of the total weight of the composition, 2.0% of the total weight of the composition, 2.5% of the total weight of the composition, 3.0% of the total weight of the composition, 3.5% of the total weight of the composition, 4.0% of the total weight of the composition, 4.5% of the total weight of the composition, 5.0% of the total weight of the composition, 6.0% of the total weight of the composition, 7.0% of the total weight of the composition, 7.5% of the total weight of the composition, 8.0% of the total weight of the composition, 9.0% of the total weight of the composition, 10.0% of the total weight of the composition, 12.5% of the total weight of the composition, 15.0% of the total weight of the composition, 17.5% of the total weight of the composition, 20.0% of the total weight of the composition, 22% of the total weight of the composition, 22.5% of the total weight of the composition, 25.0% of the total weight of the composition, 27% of the total weight of the composition, 27.5% of the total weight of the composition, 30.0% of the total weight of the composition, 32% of the total weight of the composition, 32.5% of the total weight of the composition, 35.0% of the total weight of the composition, 37% of the total weight of the composition, 37.5% of the total weight of the composition, 40.0% of the total weight of the composition, 42% of the total weight of the composition, 42.5% of the total weight of the composition, 45.0% of the total weight of the composition, 47% of the total weight of the composition, 47.5% of the total weight of the composition, 49% of the total weight of the composition, 49.5% of the total weight of the composition, and 50.0% of the total weight of the composition. In some examples, a composition described herein comprises a phospholipid in an amount within a range of the amounts described in this paragraph.

In one example, a composition described herein comprises a phospholipid in an amount ranging from 1 mg to 50 mg. In another example, a composition described herein comprises a phospholipid in an amount selected from the group consisting of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, and 50 mg. In some examples, a composition described herein comprises a phospholipid in an amount within a range of the amounts described in this paragraph.

### Nanoparticles

In one example, the composition further includes metal nanoparticles or mineral nanoparticles (“metal/mineral nanoparticles”). Examples include gold, silver, zinc, copper, iridium, platinum, silica, and palladium. The metal particles or mineral particles, according to embodiments herein, may be in the form of nano and/or micro or micronized particles. Further, the nanoparticles may be stabilized by one of more emulsifiers, oils, surfactants, etc. The particle size of the metal particles or mineral particles, according to embodiments herein, are nano and/or micro or micronized particles which, as used herein refers to particles in the nano meter and micrometer range. The term “nano” or “nanoparticle”, as used herein is intended to cover all such particles having nano meter and micrometer range particles. It may range between 1 nm to 1 μm, preferably 0.1 μm to 0.5 μm or 10 nm to 100 nm. Preferably, nanometer refers to particles in the size range of 1 to 100 nm, or 10 to 100 nm. Preferably, micronized refers to particles in the size range of 100 to 500 nm, or 100 to 200 nm or 200 to 400 nm. Further, the metal particles may be present or introduced in the composition in any suitable form or generally accepted form, for e.g.: colloidal, suspended, dispersed, unaggregated, etc. Colloidal forms, in some embodiments preferably refers to metal particles suspended in solution or liquid than being added to a solution or suspension, or a gel or tablet.

In one example, a composition disclosed herein comprises one or more metal/mineral nanoparticles selected from the group consisting of a nano or micronized gold, nano or micronized silver, nano or micronized zinc, nano or micronized copper, nano or micronized iridium, nano or micronized platinum, nano or micronized silica, and nano or micronized palladium.

In another example, a composition disclosed herein comprises one or more metal/mineral nanoparticles selected from the group consisting of a nano or micronized colloidal gold, nano or micronized colloidal silver, nano or micronized colloidal zinc, nano or micronized colloidal copper, nano or micronized colloidal iridium, nano or micronized colloidal platinum, nano or micronized colloidal silica, and nano or micronized colloidal palladium.

In another example, a composition disclosed herein comprises one or more metal nanoparticles in solution.

In another example, a composition disclosed herein comprises a nano or micronized metal Diferuloylmethane combination. In another example, a composition disclosed herein comprises a nano or micronized colloidal metal Diferuloylmethane combination.

In one example, the metal nanoparticles have an optimum Zeta potential. Zeta potential is generally known as the potential difference between the dispersion medium and the stationary layer of fluid attached to the particle. The Zeta potential charge of a metal nanoparticle, according to some examples herein can range from −1.0 mV to −40 mV. In another example, a composition described herein comprises a metal nanoparticle having a zeta potential selected from the group consisting of −1.0 mV, −1.5 mV, −2.0 mV, −2.5 mV, −3.0 mV, −3.5 mV, −4.0 mV, −4.5 mV, −5.0 mV, −5.5 mV, −6.0 mV, −6.5 mV, −7.0 mV, −7.5 mV, −8.0 mV, −8.5 mV, −9.0 mV, −9.5 mV, −10.0 mV, −10.5 mV, −11.0 mV, −11.5 mV, −12.0 mV, −12.5 mV, −13.0 mV, −13.5 mV, −14.0 mV, −14.5 mV, −15.0 mV, −15.5 mV, −16.0 mV, −16.5 mV, −17.0 mV, −17.5 mV, −18.0 mV, −18.5 mV, −19.0 mV, −19.5 mV, −20.0 mV, −20.5 mV, −21.0 mV, −21.5 mV, −22.0 mV, −22.5 mV, −23.0 mV, −23.5 mV, −24.0 mV, −24.5 mV, −25.0 mV, −25.5 mV, −26.0 mV, −26.5 mV, −27.0 mV, −27.5 mV, −28.0 mV, −28.5 mV, −29.0 mV, −29.5 mV, −30.0 mV, −30.5 mV, −31.0 mV, −31.5 mV, −32.0 mV, −32.5 mV, −33.0 mV, −33.5 mV, −34.0 mV, −34.5 mV, −35.0 mV, −35.5 mV, −36.0 mV, −36.5 mV, −37.0 mV, −37.5 mV, −38.0 mV, −38.5 mV, −39.0 mV, −39.5 mV and −40.0 mV. In some examples, a composition described herein comprises a metal nanoparticle having a Zeta potential within a range of the Zeta potentials described in this paragraph.

In one example, a composition described herein comprises a metal nanoparticle in an amount ranging from 2 ppm to 400 ppm. In another example, a composition described herein comprises a metal nanoparticle in an amount selected from the group consisting of 0.5 ppm, 2.0 ppm, 5.0 ppm, 7.5 ppm, 10.0 ppm, 12.5 ppm, 15.0 ppm, 17.5 ppm, 20.0 ppm, 22.5 ppm, 25.0 ppm, 27.5 ppm, about 30 ppm, 32.5 ppm, 35.0 ppm, 37.5 ppm, 40.0 ppm, 42.5 ppm, 45.0 ppm, 47.5 ppm, 50 ppm, 52.5 ppm, 55 ppm, 57.5 ppm, 60 ppm, 62.5 ppm, 65 ppm, 67.5 ppm, 70 ppm, 72.5 ppm, 75 ppm, 77.5 ppm, 80 ppm, 82.5 ppm, 85 ppm, 87.5 ppm, 90 ppm, 92.5 ppm, 95 ppm, 97.5 ppm, 100.0 ppm, 102.5 ppm, 105.0 ppm, 107.5 ppm, 110.0 ppm, 112.5 ppm, 115.0 ppm, 117.5 ppm, 120.0 ppm, 122.5 ppm, 125.0 ppm, 127.5 ppm, 130.0 ppm, 132.5 ppm, 135.0 ppm, 137.5 ppm, 140.0 ppm, 142.5 ppm, 145.0 ppm, 147.5 ppm, 150.0 ppm, 152.5 ppm, 155.0 ppm, 157.5 ppm, 160.0 ppm, 162.5 ppm, 165.0 ppm, 167.5 ppm, 170.0 ppm, 172.5 ppm, 175.0 ppm, 172.5 ppm, 175.0 ppm, 177.5 ppm, 180.0 ppm, 182.5 ppm, 185.0 ppm, 187.5 ppm, 190.0 ppm, 192.5 ppm, 195.5 ppm, 197.5 ppm or 200.0 ppm, 200.0 ppm, 202.5 ppm, 205.0 ppm, 207.5 ppm, 210.0 ppm, 212.5 ppm, 215.0 ppm, 217.5 ppm, 220.0 ppm, 222.5 ppm, 225.0 ppm, 227.5 ppm, 230.0 ppm, 232.5 ppm, 235.0 ppm, 237.5 ppm, 240.0 ppm, 242.5 ppm, 245.0 ppm, 247.5 ppm, 250.0 ppm, 252.5 ppm, 255.0 ppm, 257.5 ppm, 260.0 ppm, 262.5 ppm, 265.0 ppm, 267.5 ppm, 270.0 ppm, 272.5 ppm, 275.0 ppm, 272.5 ppm, 275.0 ppm, 277.5 ppm, 280.0 ppm, 282.5 ppm, 285.0 ppm, about 287.5 ppm, 290.0 ppm, 292.5 ppm, 295.5 ppm, 297.5 ppm, 300.0 ppm, 302.5 ppm, 305.0 ppm, 307.5 ppm, 310.0 ppm, 312.5 ppm, 315.0 ppm, 317.5 ppm, 320.0 ppm, 322.5 ppm, 325.0 ppm, 327.5 ppm, 330.0 ppm, 332.5 ppm, 335.0 ppm, 337.5 ppm, about 340.0 ppm, 342.5 ppm, 345.0 ppm, 347.5 ppm, 350.0 ppm, 352.5 ppm, 355.0 ppm, 357.5 ppm, 360.0 ppm, 362.5 ppm, 365.0 ppm, 367.5 ppm, 370.0 ppm, 372.5 ppm, 375.0 ppm, 372.5 ppm, 375.0 ppm, 377.5 ppm, 380.0 ppm, 382.5 ppm, 385.0 ppm, 387.5 ppm, 390.0 ppm, 392.5 ppm, 395.0 ppm, 397.5 ppm, and 400.0 ppm. In some examples, a composition described herein comprises a metal nanoparticle in an amount within a range of the amounts described in this paragraph.

In one example, a composition described herein comprises a metal nanoparticle in an amount ranging from 0.1 mcg to 50 mcg. In another example, a composition described herein comprises a metal nanoparticle in an amount selected from the group consisting of 0.1 mcg, 0.5 mcg, 1.0 mcg, 1.5 mcg, 2.0 mcg, 2.5 mcg, 3.0 mcg, 3.5 mcg, 4.0 mcg, 4.5 mcg, 5.0 mcg, 5.5 mcg, 6.0 mcg, 6.5 mcg, 7.0 mcg, 7.5 mcg, 8.0 mcg, 8.5 mcg, 9.0 mcg, 9.5 mcg, 10.0 mcg, 10.5 mcg, 11.0 mcg, 11.5 mcg, 12.0 mcg, 12.5 mcg, 13.0 mcg, 13.5 mcg, 14.0 mcg, 14.5 mcg, 15.0 mcg, 15.5 mcg, 16.0 mcg, 16.5 mcg, 17.0 mcg, 17.5 mcg, 18.0 mcg, 18.5 mcg, 19.0 mcg, 19.5 mcg, 20.0 mcg, 20.5 mcg, 22.0 mcg, 22.5 mcg, 23.0 mcg, 23.5 mcg, 24.0 mcg, 24.5 mcg, 25.0 mcg, 27.5 mcg, 30.0 mcg, 32.5 mcg, 35.0 mcg, 37.5 mcg, 40.0 mcg, 42.5 mcg, 45.0 mcg, 47.5 mcg, and 50.0 mcg. In some examples, a composition described herein comprises a metal nanoparticle in an amount within a range of the amounts described in this paragraph.

In one example, a composition described herein comprises a metal nanoparticle in a size ranging from 1 nm to 325 nm. In another example, a composition described herein comprises a metal nanoparticle in a size selected from the group consisting of 1 nm, 2 nm, 3 nm, 4 nm, 5 nm, 6 nm, 7 nm, 8 nm, 9 nm, 10, nm, 12.5 nm, 15.0 nm, 17.5 nm, 20.0 nm, 22.5 nm, 25.0 nm, 27.5 nm, 30 nm, 32.5 nm, 35 nm, 37.5 nm, 40.0 nm, 42.5 nm, 45.0 nm, 47.5 nm, 50.0 nm, 52.5 nm, 55.0 nm, 57.5 nm, 60.0 nm, 62.5 nm, 65.0 nm, 67.5 nm, 70.0 nm, 72.5 nm, 75.0 nm, 77.5 nm, 80.0 nm, 82.5 nm, 85.0 nm, 87.5 nm, 90.0 nm, 92.5 nm, 95.0 nm, 97.5 nm, 100.0 nm, 102.5 nm, 105.0 nm, 107.5 nm, 110.0 nm, 112.5 nm, 115.0 nm, 117.5 nm, 120.0 nm, 122.5 nm, 125.0 nm, 127.5 nm, 130.0 nm, 132.5 nm, 135.0 nm, 137.5 nm, 140.0 nm, 142.5 nm, 145.0 nm, 147.5 nm, 150.0 nm, 152.5 nm, 155.0 nm, 157.5 nm, 160.0 nm, 162.5 nm, 165.0 nm, 167.5 nm, 170 nm, 172.5 nm, 175.0 nm, 177.5 nm, 180.0 nm, 182.5 nm, 185.0 nm, 187.5 nm, 190.0 nm, 192.5 nm, 195.0 nm, 197.5 nm, 200.0 nm, 202.5 nm, 205.0 nm, 207.5 nm, 210.0 nm, 212.5 nm, 215.0 nm, 217.5 nm, 220.0 nm, 222.5 nm, 225.0 nm, 227.5 nm, 230.0 nm, 232.5 nm, 235.0 nm, 237.5 nm, 240 nm, 242.5 nm, 245.0 nm, 247.5 nm, 250.0 nm, 252.5 nm, 255.0 nm, 257.5 nm, 260.0 nm, 262.5 nm, 265.0 nm, 267.5 nm, 270 nm, 272.5 nm, 275.0 nm, 277.5 nm, 280.0 nm, 282.5 nm, 285.0 nm, 287.5 nm, 290.0 nm, 292.5 nm, 295.0 nm, 297.5 nm, 300.0 nm, 302.5 nm, 305.0 nm, 307.5 nm, 310.0 nm, 312.5 nm, 315.0 nm, 317.5 nm, 320.0 nm, 322.5 nm and 325.0 nm. In some examples, a composition described herein comprises a metal nanoparticle in a size that is within a range of the sizes described in this paragraph.

The metal nanoparticles, according to embodiments herein, are such that it is capable of being activated when illuminated with light of suitable wavelength, preferably ranging from about 600 nm to about 750 nm.

The composition may further include other generally known additive such as antiallergens, antihistamines, for e.g.: diphenhydramine hydrochloride.

### Excipients

The disclosed composition, in the various embodiments herein, may further include one or more pharmaceutical acceptable excipients. The pharmaceutical acceptable excipients include, but is not limited to, solvents, binders, emulsifiers, lubricants, carriers, oils, etc. In an embodiment, the excipient is present in an amount in the range of 0.2 wt % to 70 wt. % of the total composition.

Carriers, according to embodiments herein, include hydrophilic and/or hydrophobic solvents, compounds, etc. In an embodiment, the solvent is aqueous solvent, preferably water. In an embodiment, the carriers are selected from water soluble polymers, water oil-soluble polymer and lipophilic aqueous system. The water soluble polymers, in embodiments herein, may be at least one selected from a group consisting of polyglutamic acid, polyethylene glycol, propylene glycol, glycerol, propylene glycol esters, polyglycerol oleate polyvinyl alcohol, non-ethoxylated polymers like glyceryl stearate (and) Polyglyceryl-6 Palmitate/Succinate (and) Cetearyl Alcohol (NatraGem™ EW) and N-(2-Hydroxypropyl) methacrylamide (PHPMA). Alternatively, other generally known water soluble polymers may be also used in other embodiments. In another embodiment disclosed herein, the composition comprises of lipophilic aqueous system. Lipophilic aqueous system, in various embodiments herein, includes at least one lipophilic solvent selected from a group consisting of a triglyceride such as fractionated oil, a caprylic triglyceride, an oil containing chain fatty acid triglyceride, isopropyl myristate, isopropyl palmitate, ethyl linoleate, an ethyl oleate ester of fatty acid, propylene glycol dicaprylate, propylene glycol dilaurate and an oil containing propylene glycol fatty acid ester.

The composition, in an embodiment, includes water oil-soluble polymers. The water oil-soluble polymer, in embodiments herein, may be at least one selected from a group of polymers consisting of a polyglutamic acid, a polyethylene glycol, a propylene glycol, glycerol, a propylene glycol ester, a polyglycerol oleate polyvinyl alcohol, and a non-ethoxylated polymer.

The disclosed composition may include the carrier in a suitable amount. In an embodiment, the amount of carriers ranges from 5 wt % to 70 wt % of the composition. In an embodiment, the lipophilic solvent in the composition is present in an amount in the range of about 50 to 200 wt % of the curcuminoid. In an embodiment, the composition includes lipophilic solvent comprising a triglyceride.

Further, the composition, according to embodiments herein, may include one or more emulsifiers, preferably non-ionic oil-in-water emulsifier. In an embodiment, the composition includes at least one non-ionic oil-in-water emulsifier selected from a group consisting of a polyoxyethylene product of a hydrogenated vegetable oil, a polyethoxylated source oil, a polyethoxylated hydrogenated plant source oil, a polyoxyethylene-sorbitan-fatty acid ester and a polyoxyethylene castor oil derivative.

The composition may comprise about 30 wt % to 45 wt % of the non-ionic oil-in-water emulsifier. In an embodiment, the composition comprises emulsifier in the range of about 30%, 35%, 40% or 45% of the total composition.

The composition, according to embodiments herein, may further include a solvent. In an embodiment, the composition includes at least one solvent selected from a group consisting of aqueous water, a polyethylene glycol, glycerol, a polypropylene glycol, propylene glycol, and a polysorbate.

The composition, according to embodiments herein, may further include one or more adsorbents. Further in an embodiment disclosed herein, the adsorbent is selected from a group consisting of a polyethylene glycol, a maltodextrin, a soluble starch, a hydrolyzed starch, a fiber, a water-soluble oligo saccharide, a chicory, a dextran, a cellulose, a copolymer of polyvinylpyrrolidone, a sugar alcohol, a sorbitol, a xylitol, a mannitol and a cellulose derivative. Further, the composition in some embodiments may include cellulose derivative such as hydroxypropylmethyl cellulose, a hydroxypropyl cellulose, a cyclodextrine or combinations thereof.

The composition, according to embodiments herein may further include one or more diluents, fillers and/or binders. In an embodiment, the composition includes at least one sugar, preferably polyols. Examples of sugars include, but is not limited to, mannitol, a sorbitol; sugars such as lactose, sodium saccharine, Acesulfame K, Alitame, Aspartame, Dextrose, Fructose, Galactose, Inulin, Isomalt, Lactitol, Maltitol, Maltose, Mannitol, Neohesperidin dihydrochalcone, Saccharin, Sodium cyclamate, Sorbitol, Sucralose, Sucrose, Tagatose, Thaumatin, Trehalose and Xylitol, or combinations thereof.

The composition in other embodiments may further include other suitable and pharmaceutically acceptable excipients such as stabilizer, a surfactant, a plasticizer, a lubricant, a reducing agent, a buffering agent, a sweetening agent, a base, a corrigent, a suspending agent, an antioxidant, a polish, a coating, a wetting agent, a gelling agent, a wet modifier, an antifoaming agent, a refrigerative agent, a coloring matter, a flavoring agent, a perfume, a sugar coating agent, an isotonizing agent, a softener, a foaming agent, a pH modifier, an anti-frothing agent, a flavoring agent, a preservatives, a dispersing agent, a disintegrator, a fragrance, a desiccant, an antiseptics, an preservative, a solubilizing agent, a solubilizer, a superplasticizer, an antistatic agent, an extender, a moisturizing agent, or combinations thereof. Those in the art will understand that a number of variations may be made in the disclosed embodiments, all without departing from the scope of the invention. Such variations are understood to fall within the scope of the appended claims.

In an embodiment disclosed herein, the composition further comprises glycerin in an amount ranging from about 0.5% to 30% of the total weight of the composition. The amount of glycine in the composition may be 0.5% 1.0%, 1.5%, 2.0%, 2.5%, 3.0%, 3.5%, 4.0%, 4.5%, 5.0%, 6.0%, 7.0%, 8.0%, 9.0%, 10.0%, 12.5%, 15.0%, 17.5%, 20.0%, 22.5%, 25.0%, 27.5% or 30.0% of the total weight of the composition.

In an embodiment disclosed herein, the composition further comprises polysorbate in an amount ranging from about 0.5% to 30% of the total weight of the composition. The amount of polysorbate in the composition may be 0.5% 1.0%, 1.5%, 2.0%, 2.5%, 3.0%, 3.5%, 4.0%, 4.5%, 5.0%, 6.0%, 7.0%, 8.0%, 9.0%, 10.0%, 12.5%, 15.0%, 17.5%, 20.0%, 22.5%, 25.0%, 27.5% or 30.0% of the total weight of the composition.

In an embodiment disclosed herein, the composition further comprises polyethylene glycol (PEG) or similar polymer structure with native as well conjugated form in an amount ranging from 0.5% to 25% of the total weight of the composition. The amount of PEG in the composition may be 0.5% 1.0%, 1.5%, 2.0%, 2.5%, 3.0%, 3.5%, 4.0%, 4.5%, 5.0%, 6.0%, 7.0%, 8.0%, 9.0%, 10.0%, 12.5%, 15.0%, 17.5%, 20.0%, 22.5% or 25.0% of the total weight of the composition. Further in an embodiment disclosed herein, the PEG can include, without limitation PEG 100, PEG 200, PEG 300, PEG 400, PEG 500, PEG 600, PEG 700, PEG 800, PEG 900, PEG 1000, PEG 1100, PEG 1200, PEG 1300, PEG 1400, PEG 1500, PEG 1600, PEG 1700, PEG 1800, PEG 1900, PEG 2000, PEG 2100, PEG 2200, PEG 2300, PEG 2400, PEG 2500, PEG 2600, PEG 2700, PEG 2800, PEG 2900, PEG 3000, PEG 3250, PEG 3350, PEG 3500, PEG 3750, PEG 4000, PEG 4250, PEG 4500, PEG 4750, PEG 5000, PEG 5500, PEG 6000, PEG 6500, PEG 7000, PEG 7500, PEG 8000, PEG 8500, PEG 9000, PEG 9500, PEG 10,000, PEG 11,000, PEG 12,000, PEG 13,000, PEG 14,000, PEG 15,000, PEG 16,000, PEG 17,000, PEG 18,000, PEG 19,000, or PEG 20,000. In a preferred embodiment, the PEG is PEG 200. In a more preferred embodiment, the PEG is PEG 400.

In an embodiment disclosed herein, the composition further comprises mannitol in an amount ranging from 10% to 60% of the total weight of the composition. The amount of mannitol in the composition may be 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 57.5% or 60% of the total weight of the composition.

In an embodiment disclosed herein, the composition further comprises corn starch in an amount ranging from 20% to 50% of the total weight of the composition. The amount of corn starch in the composition may be 20%, 22.5%, 25%, 27.5%, 30%, 32.5%, 35%, 37.5%, 40%, 42.5%, 45%, 47.5% or 50% of the total weight of the composition.

The composition may further comprises one or more pharmaceutically acceptable gelling agents, preferably suitable for achieve semi solid dosage forms such as tragacanth; pectin; starch; carbomer; sodium alginate; cellulose derivatives; gums such as guar gum, xanthan gum, acacia gum, etc; olyvinyl alcohol clays; and gelatin, such as fish gelatin, porcine gelatin, bovine gelatin, etc. In an embodiment, the composition comprises fish gelatin. In another embodiment, the composition comprises bovine gelatin.

Gelatin may be included in the composition in a suitable amount, preferably ranging from 2% to 40% of the total weight of the composition. In an embodiment, the amount of gelatin is about 2.0%, 2.5%, 3.0%, 3.5%, 4.0%, 4.5%, 5.0%, 6.0%, 7.0%, 8.0%, 9.0%, 10.0%, 12.5%, 15.0%, 17.5%, 20.0%, 22.5%, 25.0%, 27.5%, 30.0%, 32.5%, 35%, 37.5% or 40% of the total weight of the composition.

In an embodiment disclosed herein, the composition further comprises sucralose in an amount ranging from 0.1 mg to 5.0 mg. The amount of sucralose in the composition may be 0.1 mg, 0.15 mg, 0.2 mg, 0.25 mg, 0.5 mg, 1.0 mg, 1.5 mg, 2.0 mg equivalent, 2.5 mg, 3 mg, 3.5 mg, 4.0 mg, 4.5 mg, or 5.0 mg.

In an embodiment disclosed herein, the composition further comprises glycine in an amount ranging from 0.5% to 10% of the total weight of the composition. The amount of glycine in the composition may be 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5% or 10.0%, of the total weight of the composition.

In an embodiment disclosed herein, the composition further comprises mint flavor present in an amount ranging from 0.025% to 1.0% of the total weight of the composition. The amount of mint flavor in the composition may be 0.025%, 0.05%, 0.1%, 0.5%, 0.75% or 1.0% of the total weight of the composition.

In an embodiment disclosed herein, the composition further comprises xanthan gum in an amount ranging from 1% to 60% of the total weight of the composition. The amount of xanthan gum in the composition bypassing first phase metabolism may be 1%, 2.5%, 5.0%, 7.5%, 10%, 12.5%, 15.0%, 17.5%, 20.0%, 22.5%, 25.0%, 27.5%, 30.0%, 32.5%, 35%, 37.5%, 40%, 42.5%, 45%, 47.5%, 50%, 52.5%, 57.5% or 60% of the total weight of the composition.

In an embodiment disclosed herein, the composition further comprises Carbopol 934 in an amount ranging from 1% to 50% of the total weight of the composition. The amount of Carbopol 934 in the composition may be 1%, 2.5%, 5.0%, 7.5%, 10%, 12.5%, 15.0%, 17.5%, 20.0%, 22.5%, 25.0%, 27.5%, 30.0%, 32.5%, 35%, 37.5%, 40%, 42.5%, 45%, 47.5% or 50% of the total weight of the composition.

In an embodiment disclosed herein, the composition further comprises guar gum in an amount ranging from 0.1% to 20% of the total weight of the composition. The amount of guar gum in the composition may be 0.1%, 0.25%, 0.5%, 1.0%, 1.5%, 2.0%, 2.5%, 3.0%, 3.5%, 4.0%, 4.5%, 5.0%, 6.0%, 7.0%, 8.0%, 9.0%, 10.0%, 12.5%, 15.0%, 17.5%, 20.0% of the total weight of the composition.

In an embodiment disclosed herein, the composition further comprises Carbopol 934 and guar gum in a ratio of about 5:1. In a preferred embodiment, the ratio of Carbopol 934 to guar gum in a composition described herein is about 4:1. In a more preferred embodiment, the ratio of Carbopol 934 to guar gum in a composition described herein is about 3:1.

In an embodiment disclosed herein, the composition in a liquid or a solution form comprises a total volume ranging from about 0.1 ml to about 25 ml. The total volume of the composition in a liquid or a solution form described herein may be 0.1 ml, 0.2 ml, 0.3 ml, 0.4 ml, 0.5 ml, 0.6 ml, 0.7 ml, 0.8 ml, 0.9 ml, 1.0 ml, 1.25 ml, 1.5 ml, 1.75 ml, 2.0 ml, 2.25 ml, 2.5 ml, 2.75 ml, 3.0, 3.25 ml, 3.5 ml, 3.75 ml, 4.0 ml, 4.25 ml, 4.5 ml, 4.75 ml, 5.0 ml, 5.25 ml, 5.5 ml, 5.75 ml, 6.0 ml, 6.25 ml, 6.5 ml, 6.75 ml, 7.0 ml, 7.25 ml, 7.5 ml, 7.75 ml, 8.0 ml, 8.25 ml, 8.5 ml, 8.75 ml, 9.0 ml, 9.25 ml, 9.5 ml, 9.75 ml, 10.0 ml, 10.25 ml, 10.5 ml, 10.75 ml, 11.0 ml, 11.25 ml, 11.5 ml, 11.75 ml, 12.0 ml, 12.25 ml, 12.5 ml, 12.75 ml, 13.0 ml, 13.25 ml, 13.5 ml, 13.75 ml, 14.0 ml, 14.25 ml, 14.5 ml, 14.75 ml, 15.0 ml, 15.25 ml, 15.5 ml, 15.75 ml, 16.0 ml, 16.25 ml, 16.5 ml, 16.75 ml, 17.0 ml, 17.25 ml, 17.5 ml, 17.75 ml, 18.0 ml, 18.25 ml, 18.5 ml, 18.75 ml, 19.0 ml, 19.25 ml, 19.5 ml, 19.75 ml, 20.0 ml, 20.25 ml, 20.5 ml, 20.75 ml, 21.0 ml, 21.25 ml, 21.5 ml, 21.75 ml, 22.0 ml, 22.25 ml, 22.5 ml, 22.75 ml, 23.0 ml, 23.25 ml, 23.5 ml, 23.75 ml, 24.0 ml, 24.25 ml, 24.5 ml, 24.75 ml or 25.0 ml.

In an embodiment disclosed herein, the composition is in the form of a solid or a gel with a total weight ranging from 25 mg to 150 mg. The total volume of the composition in a solid or a gel form described herein may be 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg or 150 mg.

All weight percentages are based on the total weight of the composition. However, it is apparent that slight variations and modifications in the amount of the ingredients may be practiced without otherwise departing from the intended function of the disclosed composition.

In certain embodiment, the disclosure contemplates composition comprising various combination of components such as photosensitizers, metal particles, phospholipids, excipients, carriers etc, are contemplated. In an embodiment, the composition comprises Phenothiazinium, nano or micronized colloidal metal nanoparticles, non-hydrogenated phosphatidylcholine sunflower, glycerin, PEG 400 and purified water. In another embodiment, the composition comprises Phenothiazinium, Diferuloylmethane, nano or micronized silver, non-hydrogenated phosphatidylcholine sunflower, glycerin, PEG 400 and purified water. In yet another embodiment, the composition comprises of Phenothiazinium, non-hydrogenated phosphatidylcholine sunflower, glycerin, PEG 400 and purified water.

In an embodiment, the composition comprises of Phenothiazinium, Diferuloylmethane, non-hydrogenated phosphatidylcholine sunflower, glycerin, PEG 400 and purified water. In another embodiment, the composition comprises of Phenothiazinium, non-hydrogenated phosphatidylcholine sunflower, glycerin, PEG 400 and purified water. In another embodiment, the composition comprises of Phenothiazinium, Diferuloylmethane, nano or micronized silver, non-hydrogenated phosphatidylcholine, glycerin, PEG 400 and purified water. In another embodiment, the composition comprises of Phenothiazinium, nano or micronized colloidal metal nanoparticles, hydrogenated phosphatidylcholine, glycerin, PEG 400 and purified water.

In another embodiment, the composition comprises of Phenothiazinium, nano or micronized silver, hydrogenated phosphatidylcholine, glycerin, PEG 400 and purified water. In another embodiment, the composition comprises of Phenothiazinium, hydrogenated phosphatidylcholine, glycerin, PEG 400 and purified water. In another embodiment, the composition comprises of Phenothiazinium, hydrogenated phosphatidylcholine soybean, glycerin, PEG fatty chain and purified water.

Further, in another embodiment, the composition comprises of Phenothiazinium, nano or micronized metal nanoparticles, hydrogenated phosphatidylcholine soybean, glycerin, PEG 400 and purified water. In another embodiment, the composition comprises of Phenothiazinium, nano or micronized metal nanoparticles, non-hydrogenated phosphatidylcholine soybean, glycerin, PEG 400 and purified water. In another embodiment, the composition comprises of Phenothiazinium, nano or micronized metal nanoparticles, non-hydrogenated phosphatidylcholine soybean, glycerin, PEG 400 and purified water. In yet another embodiment, the composition comprises of Phenothiazinium, nano or micronized metal nanoparticles, non-hydrogenated phosphatidylcholine sunflower, mannitol, corn starch and purified water.

Further, in an embodiment, the composition comprises of Phenothiazinium, Diferuloylmethane, nano or micronized silver, non-hydrogenated phosphatidylcholine sunflower, mannitol, gelatin-fish, corn starch and purified water. In another embodiment, the composition comprises of Phenothiazinium, nano or micronized silver, phosphatidylcholine sunflower, mannitol, gelatin fish, mint flavor, sucralose and purified water. In another embodiment, the composition comprises of Phenothiazinium, nano or micronized silver, non-hydrogenated phosphatidylcholine, mannitol, bovine gelatin, mint flavor and sucralose. In another embodiment, the composition comprises of Phenothiazinium, nano or micronized metal nanoparticles, hydrogenated phosphatidylcholine, glycine, gelatin-fish, mannitol, mint flavor, sucralose and purified water.

In an embodiment, the composition comprises of Phenothiazinium, nano or micronized metal nanoparticles, phosphatidylcholine, xanthan gum and purified water.

In another embodiment, the composition comprises of Phenothiazinium, Diferuloylmethane, nano or micronized metal nanoparticles, phosphatidylcholine, Carbopol-934, guar gum and purified water. Further, in an embodiment, the composition comprises of Phenothiazinium, nano or micronized metal nanoparticles, phosphatidylcholine, Carbopol-934 and purified water.

The composition, according to embodiments herein is preferably a clear solution liquid composition capable of remaining in clear solution form without precipitation or separation of particles for a minimum period of about 24 hours. In an embodiment, the disclosed composition is a homogenous composition. The disclosed composition is capable of bypassing first pass metabolism, preferably as its ability as a homogenous composition to result into a clear and transparent solution, without precipitation or separation of particles for a minimum period of 20 minutes when added to water in ratio of 1:200 and above. Embodiments herein provide stable compositions. The composition, according to an embodiment herein, is stabilized such that the composition when stored at normal ambient conditions (eg: at temperature 25±2 degrees Celsius and Relative Humidity 60%+5) is stable in aqueous phase, after about 1:200 dilution, for a period of up to 4 months. The composition is suitable for sublingual administration, having no or reduced phase separation.

### Dosage Form

The composition, according to embodiments herein, may further be formulated into any suitable dosage form. Various dosage forms of the composition are contemplated in the present disclosure. Generally known dosage forms, including, but not limited to, liquid forms including colloids; injectables, emulsion, suspensions, dispersion, etc.; solid forms such as capsules, lozenges, powder, lyophilized cakes, granules; semisolid forms including gel, cream, lotions, etc.; gaseous forms including aerosols. In an embodiment, the dosage form is suited for oromucosal route of administration e.g., buccal, sublingual, etc. In another embodiment, the composition is administered intraorally and the components be prepared for uptake in a manner that makes the composition available in therapeutically effective amounts. As such, they may also be prepared as water soluble compositions, deliverable in encapsulated, or in a manner suitable for time release, delayed release or any manner typically used for delivery of pharmaceuticals, nutraceuticals or vitamins, etc. In some embodiments, the composition may also be administered intranasally and intravenously

In an embodiment, the disclosed composition is formulated in the form of tablets. Generally known methods of formulating/processing compositions may be used to formulate the desired dosage forms. In an embodiment, the disclosed composition is formulated in the form of tablets, preferably with a total weight ranging from about 40 mg to 100 mg. Further in an embodiment, the composition is in the form of orally soluble tablet, preferably capable of dissolving within 2 to 30 minutes, preferably 5 to 10 minutes, under tongue or in buccal cavity. In an embodiment, orally soluble tablet is a lozenge.

In an embodiment, the composition is an oral liquid dosage form e.g., solution, suspension, dispersion, etc. In a preferable embodiment, the oral solution has a total volume ranging from about 0.1 ml to 25 ml.

In other embodiment, the composition is a semisolid dosage form. The semisolid form may be selected from the group consisting of liquid, solution, suspension, lotion, cream, ointment, gel, paste, aerosol foam or spray. In an embodiment, the semisolid form comprises a total weight ranging from 0.25 mg to 50 mg.

In an embodiment, the composition is an oral solid dosage form e.g., tablets, capsule, lozenges, powder, granules, pellets, etc, In an embodiment, the composition is an Orally Disintegrating dosage form selected from Tablet (ODT) or capsule or tablet in capsule, or a hard sugar boiled lozenge. In an embodiment, the orally disintegrating dosage form is such that it dissolves in mouth within a period of 5 seconds to 30 minutes, preferably from 30 seconds to 2 minutes both orally or sublingually. It will be apparent to those skilled in the art that many modifications, both to materials and methods, may be practiced without departing from the scope of the claims herein.

The embodiments herein may be packaged into suitable form. Processing techniques and type of packaging would vary depending on dosage forms. For example, tablets are generally compressed by tablet pressers under pressure; capsules involve capsule filling operations; granules generally involve wet granulation techniques, etc. Example of packaging include ampoules, sachet packaging, vials, dosing doppler, containers, strip package, blister packaging, syringe. In an embodiment, blister packaging is used.

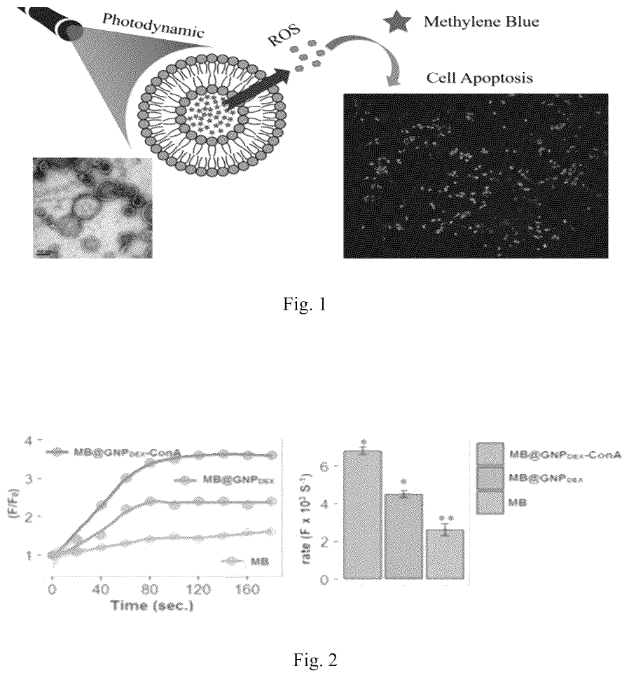

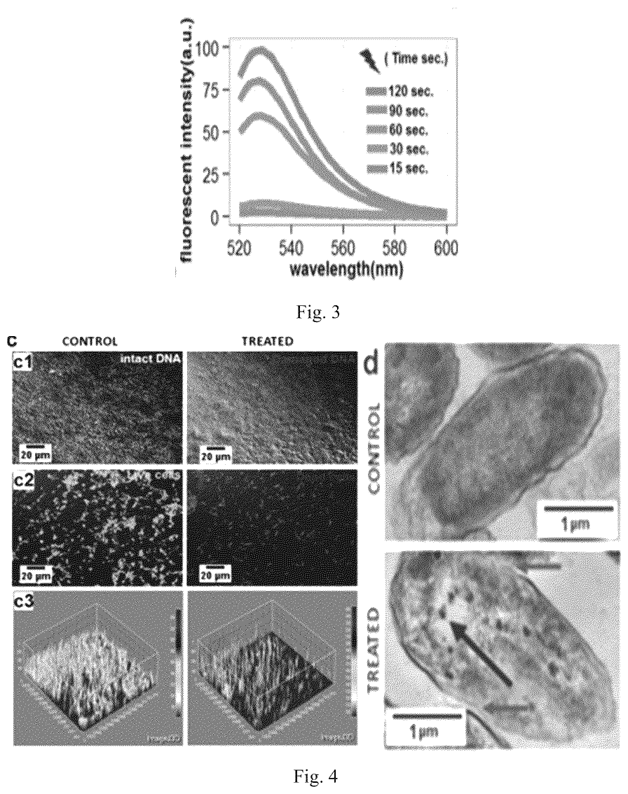

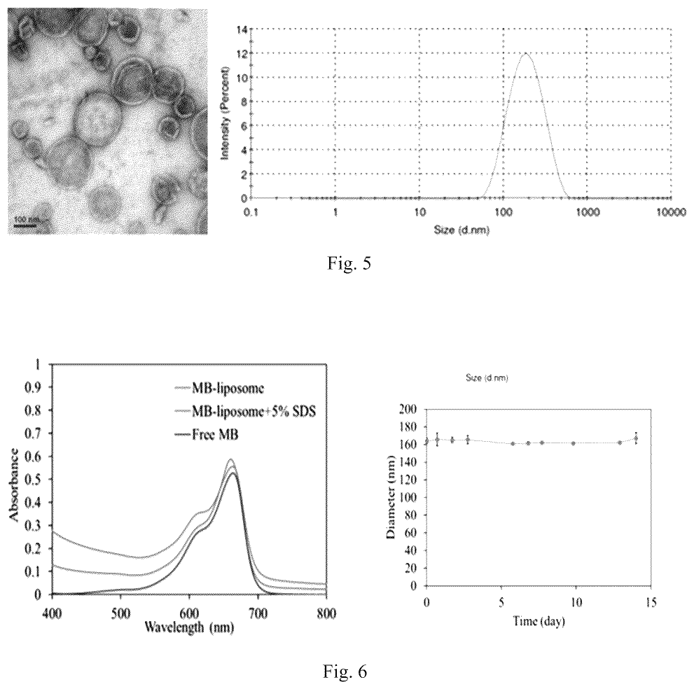

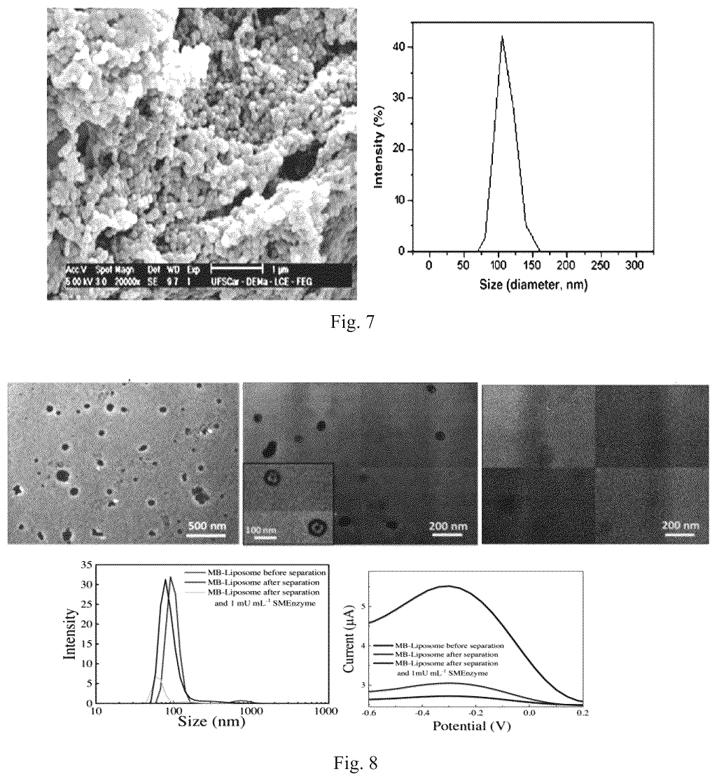

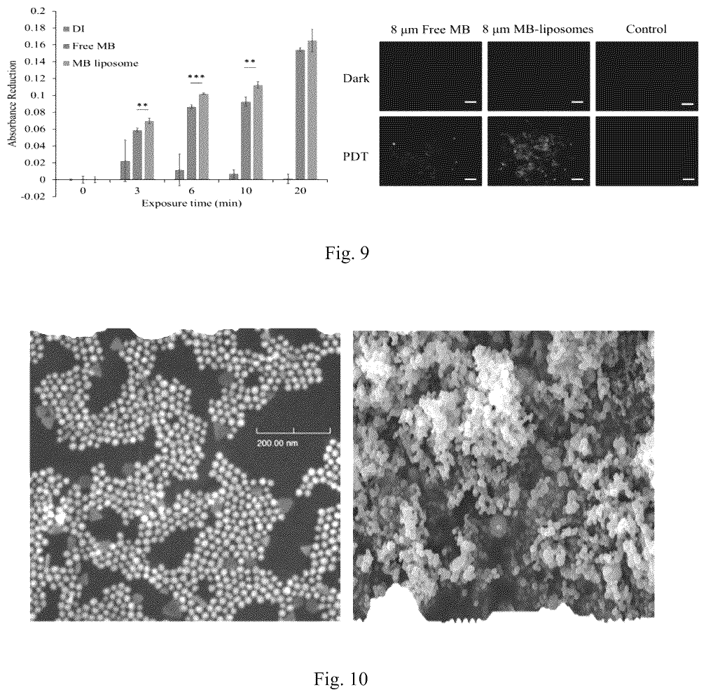

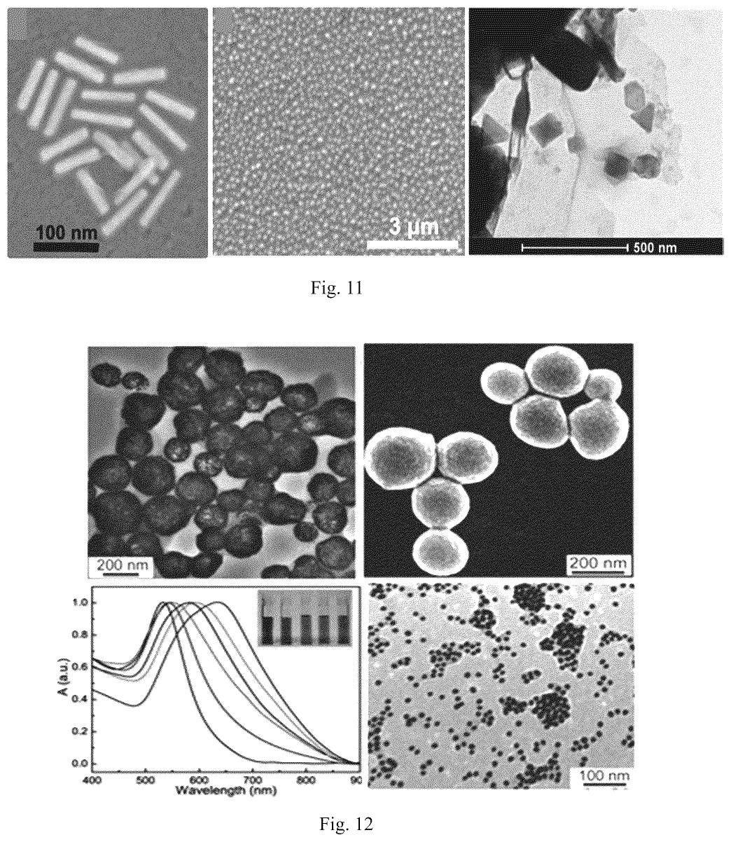

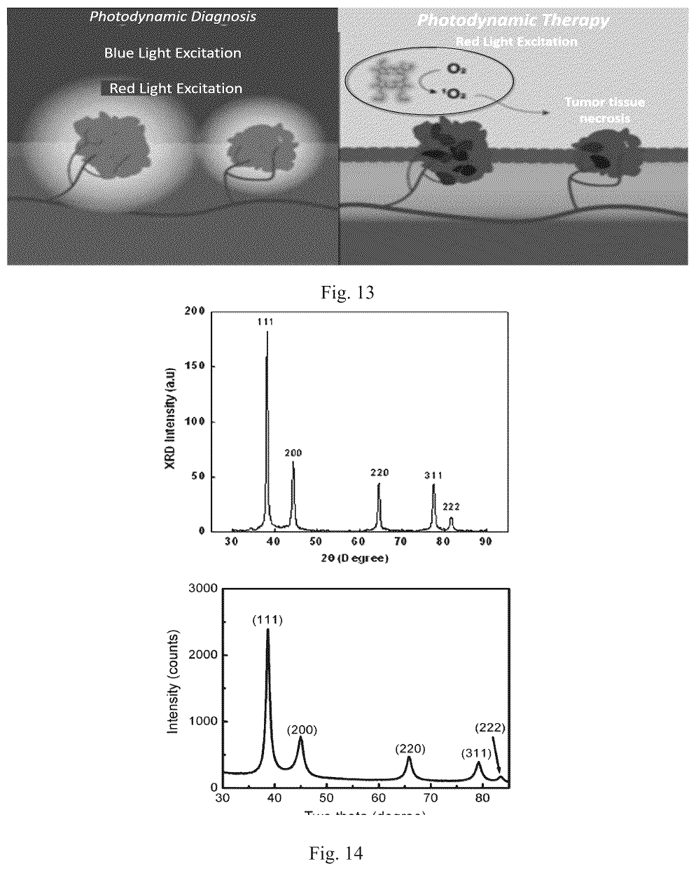

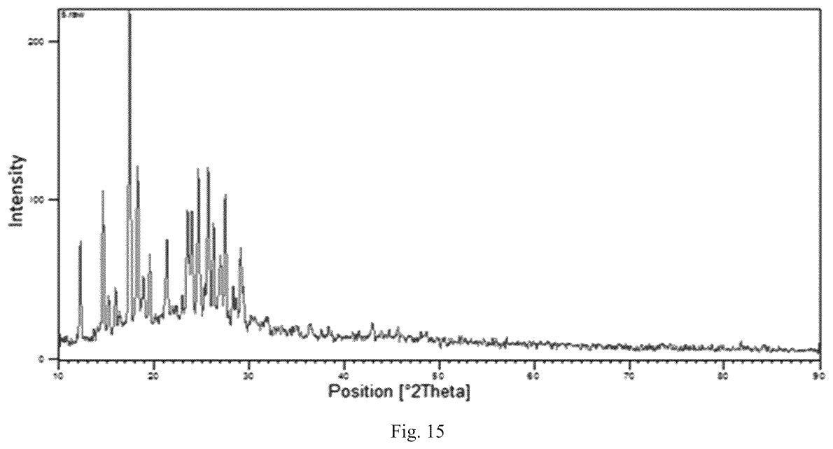

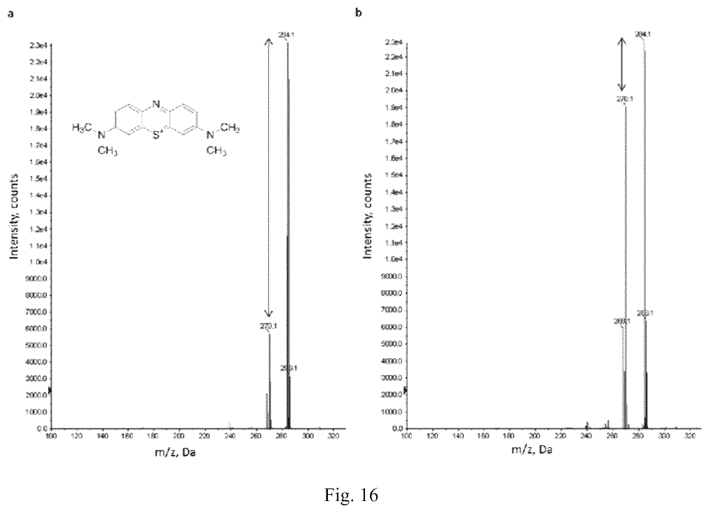

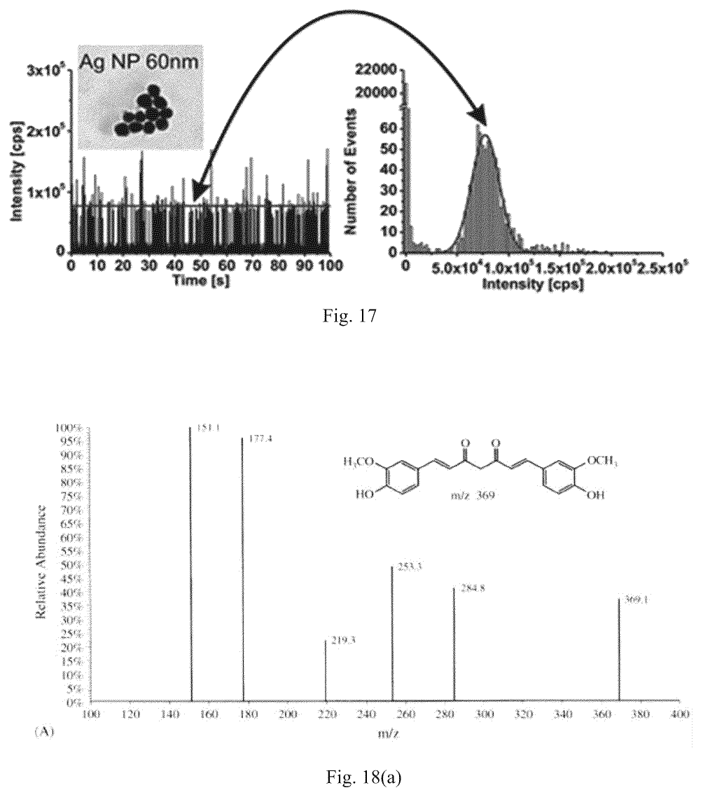

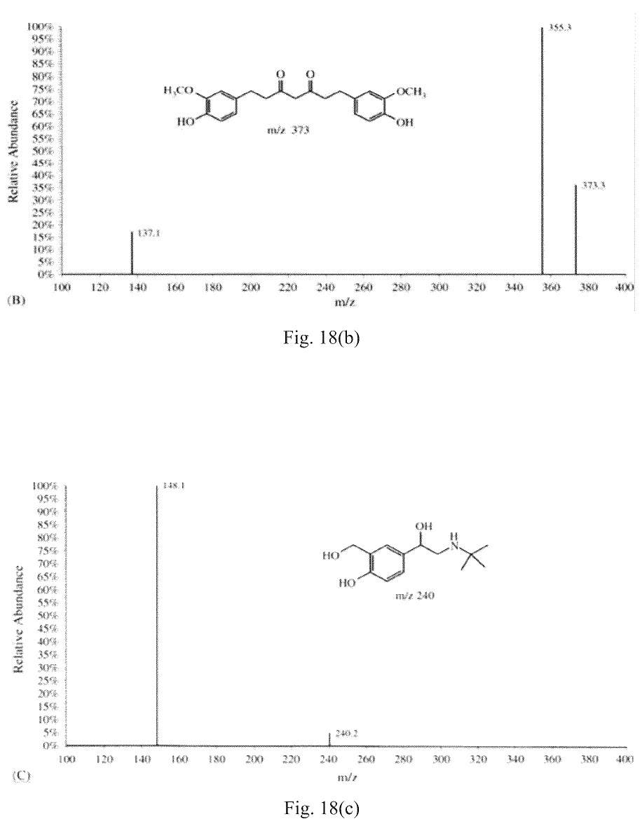

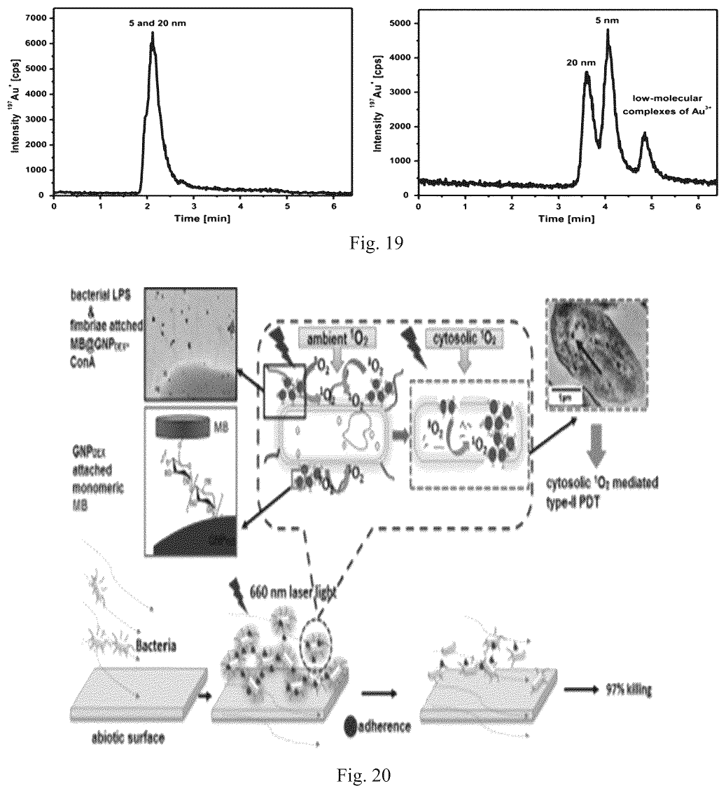

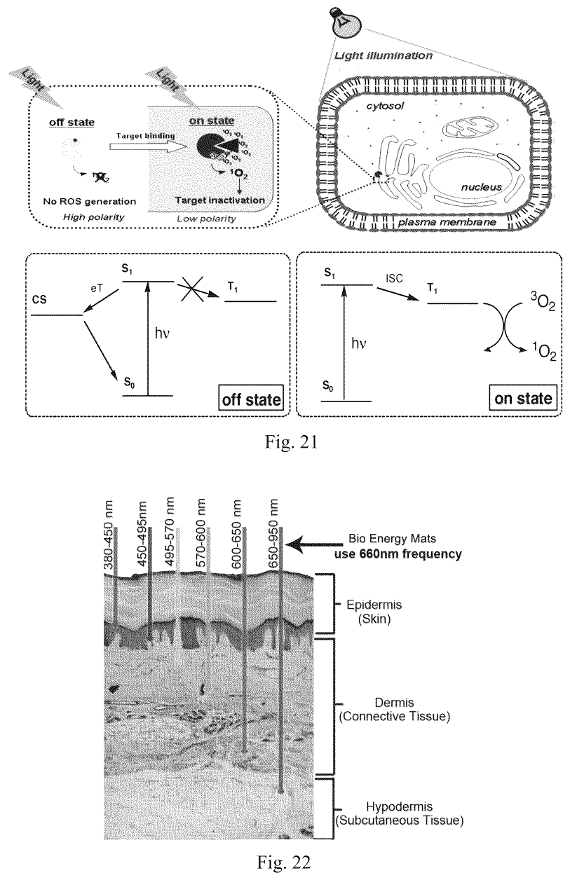

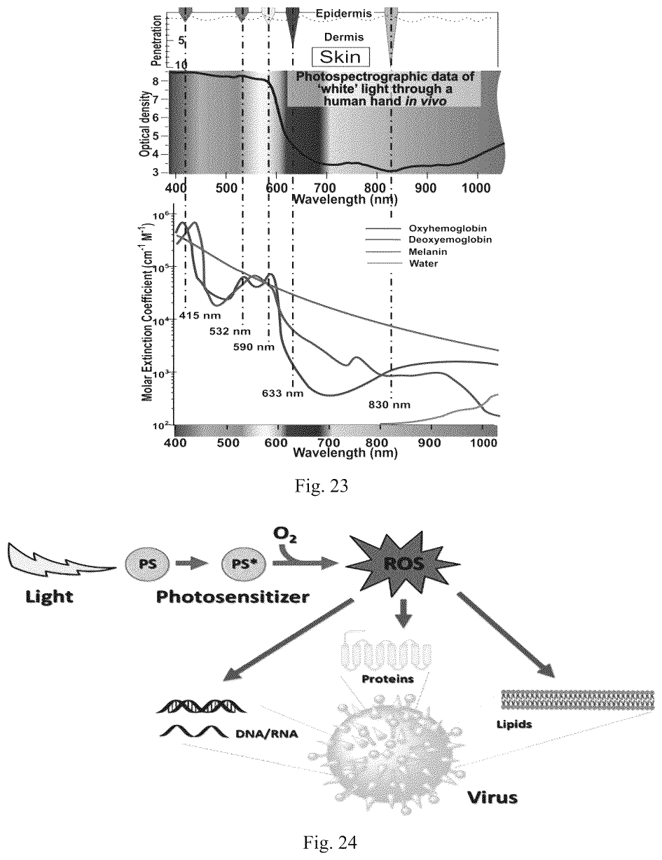

Referring now to the figures, FIG. 1 is an illustration of the cellular mechanism of action of photosensitizer. FIG. 2 are graphs depicting fluorescence intensity and rate of fluorescence of MB conjugated with gold nanoparticles. FIG. 3 depicts a graph showing increment in fluorescence intensity by Phenothiazinium category molecule. FIG. 4 are images from TEM, SEM, and Confocal Laser Scanning Microscopy (CLSM) analysis showing photo-cytotoxicity by phenothiazinium category molecule. ROS probes and PCA analysis showed the cytoplasmic O2 is antimicrobial agent. The Methylene Blue Gold Nanoparticle didn't show any photo-cytotoxicity against mammalian cells. FIG. 5 depicts TEM image of Methylene blue-liposomes and a graph showing Dynamic light scattering (DLS) measurement of MB-liposomes. FIG. 6 are UV-Vis's spectral profiles of free MB, MB-liposomes, and MB-liposomes with Nano Gold & silver 4.5 mcg & 6.5 mcg respectively, graph depicting stability of composition-hydrodynamic diameter of MB-liposomes over 14 days. FIG. 7 are TEM images of Phenothiazinium category molecule and a graph showing nano particle size distribution of 60 nm to 140 nm and its intensity of absorbance. FIG. 8 are images depicting liposome entrapped Phenothiazinium category molecule, Phenothiazinium category molecule-liposome after removal of coating, and enzymatic treatment of Phenothiazinium category molecule-liposome. FIG. 9 are graph showing 24-h cell viability (by MTT assay) of free MB and MB-liposomes and Distilled Water (DI) as control at different concentrations with dark treatment and photodynamic therapy (PDT) treatment, while the images depict free MB and MB-liposomes at 8 μm concentration and Distilled Water (DI) with dark treatment and photodynamic therapy (PDT) treatment. FIG. 10 depicts SEM images of Curcumin nanoparticles and silver & gold nanoparticles. FIG. 11 depicts SEM images of colloidal gold nanorods. FIG. 12 are images depicting Phenothiazinium category molecule, Phenothiazinium category molecule liposomal form, oxidation rate, Phenothiazinium category molecule liposomal form after sonification. FIG. 13 is a pictorial representation of blue light excitation of Liposomal-MB and red-light excitation of Liposomal-MB respectively. Under blue light, Liposomal-MB is excited and emits red fluorescence. Its detection can guide the surgical resection of the fluorescent tissues. By exciting MB with red light, reactive oxygen species such as singlet O2 are formed through photochemical reactions and energy transfer. These cause oxidative damage to vital cellular components such as lipids, proteins and nucleic acids, culminating in massive tumor cell death. FIG. 14 is a graph showing X ray diffraction pattern of Colloidal silver nanoparticles. FIG. 15 is a graph showing X ray diffraction pattern of pure curcumin. A solution of 5 mM methylene blue in D2O was irradiated overnight at 633 nm and ESI analyses were performed. FIG. 16 depicts ESI mass spectrum of methylene blue before and after overnight irradiation respectively. FIG. 17 depicts graph showing nano silver mass spectrometry pattern. FIG. 18 depicts Mass spectra Pattern of Curcumin; wherein 18(a) is mass spectra of curcumin; 18(b) is mass spectra of hydroxylated curcumin, while 18(c) is mass spectra of daughter ion of curcumin. FIG. 19 depicts graphs showing absorption spectra of colloidal gold ion of 5 nm and 20 nm and colloidal gold with low molecular complex respectively. FIG. 20 is a schematic overview of photo inactivation of multidrug resistant microbes by monomeric methylene blue conjugated gold nanoparticles. FIG. 21 is a schematic representation depicting tailoring photosensitive ROS for advance photodynamic therapy while the lower schematic representation is a Jablonski diagram showing the photo chemical mechanism. FIG. 22 is a schematic representation of Tissue penetration depths of various wavelengths. FIG. 23 is a graph with the central image shows photo spectrometric data measured from penetration through a human hand in vivo Based on the computer-derived trace on that part of the figure, the upper section illustrates relative penetration of selected wavelengths into the skin. Coupled with these, the lower section shows the absorption spectra of some biological chromophores, or targets, namely melanin, blood and water. Note the wavelength selectivity in these chromophores, and how that helps to determine the depth of penetration of different wavelengths into a living target as well as determining the target itself. FIG. 24 is a schematic representation of general action mechanism of photodynamic applications on viruses. The PDI of viruses shares the general action mechanism of photodynamic applications: the irradiation of a dye with light and the subsequent generation of ROS which are the effective phototoxic agents damaging virus targets by reacting with viral nucleic acids, lipids and proteins.

The present disclosure provides various processes for preparing a composition described herein.

In one embodiment described herein, the process for preparing a composition comprises liquid mixing methylene blue with one or more ingredients selected from (i) purified water, (ii) phospholipid selected from the group consisting of phosphatidylcholine, lyso-phophatidylcholine, and a combination thereof, (iii) nano or micronized gold and/or nano or micronized silver, (iv) PEG, (v) glycerin, (vi) mannitol, (vii) corn starch, (viii) fish gelatin, (ix) bovine gelatin, (x) mint flavor, (xi) sucralose, (xii) zanthem gum, (xiii) Carbopol 934, and (xiv) gaur gum.

In yet another embodiment described herein, the process for preparing a composition comprises liquid mixing methylene blue with one or more ingredients selected from (i) purified water, (ii) phospholipid selected from the group consisting of phosphatidylcholine, lyso-phophatidylcholine, and a combination thereof, (iii) nano or micronized gold and/or nano or micronized silver, (iv) PEG, (v) glycerin, (vi) mannitol, (vii) corn starch, (viii) fish gelatin, (ix) bovine gelatin, (x) mint flavor, (xi) sucralose, (xii) zanthem gum, (xiii) Carbopol 934, and (xiv) gaur gum, and (xv) polysorbate derivatives.

In yet another embodiment described herein, the process for preparing a composition comprises liquid mixing methylene blue with one or more ingredients selected from (i) purified water, (ii) phospholipid selected from the group consisting of phosphatidylcholine, lyso-phophatidylcholine, and a combination thereof, (iii) nano or micronized gold and/or nano or micronized silver, (iv) PEG, and (v) glycerin.

In yet another embodiment described herein, the process for preparing a composition comprises liquid mixing methylene blue with one or more ingredients selected from (i) purified water, (ii) phospholipid selected from the group consisting of phosphatidylcholine, lyso-phophatidylcholine, and a combination thereof, (iii) nano or micronized gold and/or nano or micronized silver, (iv) PEG, and (v) glycerin.

In one aspect of an embodiment described herein, the process for preparing a composition comprises a step of liquid mixing methylene blue with purified water to obtain diluted methylene blue.

In yet another aspect of an embodiment described herein, the process for preparing a composition comprises a step of liquid mixing glycerin with purified water to obtain diluted glycerin.

In yet another aspect of an embodiment described herein, the process for preparing a composition comprises a step of liquid mixing phospholipid with purified water to obtain diluted phospholipid.

In yet another aspect of an embodiment described herein, the process for preparing a composition comprises a step of liquid mixing diluted methylene blue with diluted glycerin to obtain a diluted mixture of methylene blue and glycerin.

In yet another aspect of an embodiment described herein, the process for preparing a composition comprises a step of liquid mixing methylene blue, glycerin, and diluted water to obtain a diluted mixture of methylene blue and glycerin.

In yet another aspect of an embodiment described herein, the process for preparing a composition comprises a step of liquid mixing diluted methylene blue with diluted phospholipid to obtain a diluted mixture of methylene blue and phospholipid.

In yet another aspect of an embodiment described herein, the process for preparing a composition comprises a step of liquid mixing methylene blue, phospholipid, and diluted water to obtain a diluted mixture of methylene blue and phospholipid.

In yet another aspect of an embodiment described herein, the process for preparing a composition comprises a step of liquid mixing diluted methylene blue, diluted glycerin, and phospholipid to obtain a diluted mixture of methylene blue, glycerin, and phospholipid.

In yet another aspect of an embodiment described herein, the process for preparing a composition comprises a step of liquid mixing methylene blue, glycerin, phospholipid, and purified water to obtain a diluted mixture of diluted methylene blue, glycerin, and phospholipid.

In yet another aspect of an embodiment described herein, the process for preparing a composition comprises a step of liquid mixing nano or micronized gold and/or nano or micronized silver with one or more of (i) diluted methylene blue, (ii) diluted glycerin, (iii) diluted mixture of methylene blue and glycerin, (iv) diluted mixture of methylene blue and phospholipid, and (v) diluted mixture of methylene blue, glycerin, and phospholipid.

In yet another aspect of an embodiment described herein, the process for preparing a composition comprises a step of liquid mixing nano or micronized colloidal gold and/or nano or micronized colloidal silver with one or more of (i) diluted methylene blue, (ii) diluted glycerin, (iii) diluted mixture of methylene blue and glycerin, (iv) diluted mixture of methylene blue and phospholipid, and (v) diluted mixture of methylene blue, glycerin, and phospholipid.

In yet another aspect of an embodiment described herein, the process for preparing a composition comprises a step of hydrating after at least one of the liquid mixing steps described above to obtain a hydrated mixture.

In yet another aspect of an embodiment described herein, the process for preparing a composition comprises a step of sonicating the hydrated mixture.

In yet another aspect of an embodiment described herein, the process for preparing a composition comprises a step of liquid mixing hydrated mixture and PEG to obtain a mixture of hydrated mixture and PEG.

In yet another aspect of an embodiment described herein, the process for preparing a composition comprises a step of sonicating the mixture of hydrated mixture and PEG.

In yet another aspect of an embodiment described herein, the process for preparing a composition comprises a step of liquid mixing one or more agents selected from the group consisting of mannitol, corn starch, gelatin-fish, bovine-fish, sucralose, flavoring, zanthem gum, Carbopol 934, and guar gum after at least one of the liquid mixing steps described in previous paragraphs.

In one embodiment disclosed herein, the process for preparing the composition comprises a phospholipid selected from the group consisting of phosphatidylcholine, lyso-phosphatidylcholine, and a mixture thereof.

In another embodiment disclosed herein, the process for preparing the composition comprises a phosphatidylcholine selected from the group consisting of hydrogenated phosphatidylcholine, non-hydrogenated phosphatidylcholine, hydrogenated sunflower phosphatidylcholine, non-hydrogenated sunflower phosphatidylcholine, hydrogenated soybean phosphatidylcholine, non-hydrogenated soybean phosphatidylcholine, egg yolk phosphatidylcholine, and hydrogenated egg yolk phosphatidylcholine.

In yet another embodiment disclosed herein, the process for preparing the composition comprises a PEG selected from the group consisting of PEG 100, PEG 200, PEG 300, PEG 400, PEG 500, PEG 600, PEG 700, PEG 800, PEG 900, PEG 1000, PEG 1100, PEG 1200, PEG 1300, PEG 1400, PEG 1500, PEG 1600, PEG 1700, PEG 1800, PEG 1900, PEG 2000, PEG 2100, PEG 2200, PEG 2300, PEG 2400, PEG 2500, PEG 2600, PEG 2700, PEG 2800, PEG 2900, PEG 3000, PEG 3250, PEG 3350, PEG 3500, PEG 3750, PEG 4000, PEG 4250, PEG 4500, PEG 4750, PEG 5000, PEG 5500, PEG 6000, PEG 6500, PEG 7000, PEG 7500, PEG 8000, PEG 8500, PEG 9000, PEG 9500, PEG 10,000, PEG 11,000, PEG 12,000, PEG 13,000, PEG 14,000, PEG 15,000, PEG 16,000, PEG 17,000, PEG 18,000, PEG 19,000, and PEG 20,000. In one of the embodiments, PEG in the process for preparing a composition according to the present disclosure can be substituted with Kolidone 30, Sorbitol or Glycerol.

In one example, the composition described herein comprises one or more selected from the group consisting of polysorbate 20, polysorbate 40, and polysorbate 60.

In one embodiment disclosed herein, the process for preparing the composition comprises preparing the composition a solution form.

In yet another embodiment disclosed herein, the process for preparing the composition comprises preparing the composition in a solid form. In one aspect of the embodiment disclosed herein, the process for preparing the composition comprises preparing the composition in the form of a fast dissolve tablet.

In one embodiment disclosed herein, the process for preparing the composition comprises preparing the composition a gel form.

In one embodiment disclosed herein, the step of liquid mixing comprises mixing for about 5 minutes to about 30 minutes, preferably for about 10 minutes to about 20 minutes, more preferably for about 15 minutes. A magnetic stirrer, for example, may be used for liquid mixing.

In one embodiment disclosed herein, the step of hydrating comprises mixing for about 30 minutes to about 180 minutes, preferably for about 45 minutes to about 150 minutes, more preferably for about 120 minutes. A magnetic stirrer, for example, may be used for the step of hydrating.

In one embodiment disclosed herein, the step of sonicating is carried out for about 5 minutes to about 30 minutes, preferably for about 10 minutes to about 20 minutes, more preferably for about 15 minutes, at an amplitude of about 5 to about 70, at either room temperature or in a cold bath environment.

The present disclosure also related to a composition produced by at least one of the processes described above.

In one embodiment described herein, the process for preparing the composition comprises the following steps: Glycerin (or acceptable similar material), Water, Methylene blue, Phospholipids (or acceptable similar material), PEG (or acceptable similar material), Polysorbate (or acceptable similar material), silver and gold are all mixed for 5 minutes to 180 minutes and continued mixing as needed. The processes used for mixing are general mixing, homogenization, sonication, and high shear mixing either in an ice bath or not (using pharmaceutical acceptable means of keeping the solution cool during mixing steps of materials) using pharmaceutical acceptable procedures. Material mixing combinations can range from Glycerin and water, Methylene blue in water, Phospholipid in water. Polysorbate in water, Silver, and Gold in a solution or colloidal then all mixed at different steps in several combinations to achieve the preferred composition.

In yet another embodiment described herein, the process for preparing the composition comprises the following steps:
a. liquid mixing glycerin, methylene blue, phosphatidylcholine, nano or micronized gold or nano or micronized colloidal gold and/or the nano or micronized silver or nano or micronized colloidal silver, and purified water,
b. adding fish gelatin or bovine gelatin, and flavor to (a)
c. blending/liquid mixing,
d. placing in tablet trays or clear blister pack followed by freeze drying, and
e. obtaining the composition in a fast dissolve tablet form.

Provided herein is a device for phototherapy and methods for treating a variety of conditions, including anti-cancer and anti-viral effects in a subject, using the device, wherein the device comprises at least 1 LED light to at least 10 LED lights having a specific wavelength ranging from about 600 nm to 750 nm.

The device for phototherapy as described herein comprises a housing having a light source, wherein the light source can emit light having an emission spectra which can activate a photoactivatable composition comprising at least one chromophore/fluorochrome which may then emit a light (fluorescence or phosphorescence) with therapeutic properties such as modulating blood flow in the treatment area, including bio-therapeutic reactive species, such as singlet oxygen, onto, into or nearby the treatment area.

Different embodiments of the device described herein broadly comprise a housing; wherein the housing comprises a printed circuit board (PCB) with a USB (type A, B, and/or C) charging port, and a light source. In some embodiments, the light source comprises at least 1 LED light to at least 10 LED lights having a wavelength of about 600 nm to 750 nm. In some embodiments, the light source comprises at least 1 LED light to at least 10 LED lights having different wavelengths ranging from about 600 nm to 750 nm.

In some embodiments, the wavelength of at least 1 LED light to at least 10 LED lights can be one or more selected from the group consisting of 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm.

In some embodiments, the at least 1 LED light to at least 10 LED lights comprise a wavelength that can be a combination of one or more selected from the group consisting of 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm.

In some embodiments, the device comprises any number of at least 1 LED light to at least 10 LED lights having wavelength in various combinations. For example, in one embodiment, the light source comprises a number of LED lights selected from the group consisting of 2 LED lights having a wavelength of 605 nm and 2 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm. In another embodiment, the number of LED lights can be selected from the group consisting of 3 LED lights having a wavelength of 605 nm and 3 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm. In another embodiment, the number of LED lights can be selected from the group consisting of 4 LED lights having a wavelength of 605 nm and 4 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm. In another embodiment, the number of LED lights can be selected from the group consisting of 5 LED lights having a wavelength of 605 nm and 5 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm.

In some embodiments of the device described herein, at least 1 LED light to at least 10 LED lights having various combinations of wavelengths can be illuminated at different intervals. The illumination of the LED lights can be manual or in a timed fashion.

In some embodiments, the device described herein comprises at least 1button to at least 6 buttons. The at least 1 button to at least 6 buttons may be used to control the device and/or the LED lights, including the intervals at which the LED lights are emitted. In one aspect of the embodiment, the housing comprises the at least 1 button to at least 6 buttons. In another aspect the device the at least 1 button to at least 6 buttons are located at a place other than the housing, however connected to the housing such that the buttons may be used to control the device and/or the LED lights, including the intervals at which the LED lights are emitted.

In certain embodiments described herein, the light source comprising the at least 1 LED light to at least 10 LED lights, can emit light within the orange range (600-635), or the red range of the electromagnetic spectrum (about 635 to about 750 nm). These emitted wavelengths together with the power density, fluence and/or bandwidths described above, can activate photosensitive compounds and/or compositions, and/or have a therapeutic effect themselves. The LEDs can carry a lighting power of using 150 to 3000 mA (milliamps) of lighting power and controlled watts. Further, The LEDs lights in the any combination detailed can pass through the skin at depths of 0.1 mm to 30.0 mm.

In certain embodiments described herein, the at least 1 LED light to at least 10 LED lights can be arranged in different positions and any order. In some embodiments of the device described herein, the distance between the at least 1 LED light to at least 10 LED lights ranges from about 0.5 mm to 15 mm.

In certain embodiment, the at least 1 LED light to at least 10 LED lights comprise RED and Yellow lights having an illuminate power and operating ranges as following:
a. At 1 to 30 Minutes: Red LED Lux: 10,000 to 100,000, Yellow LED Lux: 5,000 to 50,000.
b. At 30 to 60 Minutes: Red LED Lux: 10,000 to 100,000, Yellow LED Lux: 5,000 to 50,000. Note above is also covered by Foot Candle (Fc) conversion.
c. At 60 to 120 Minutes: Red LED Lux: 10,000 to 100,000, Yellow LED Lux: 5,000 to 50,000.
d. Note above is also covered by Foot Candle (Fc) conversion

In certain embodiments described herein, the light emitted from the light source, as well as being an activating light for a chromophore in a photoactivatable composition, may also have therapeutic benefits itself when applied to tissue e.g., antiviral properties. Therefore, it may be advantageous to use the device with a photoactivatable dosage form composition which also allows the activating light to pass therethrough to be able to irradiate the areas from where the compositions may be easily accessed. The photoactivatable dosage form composition may be substantially transparent or translucent, or otherwise optically conductive.

In some aspects of the embodiments described herein, the light emitted from the light source generates a temperature between about 40° F. to about 100° F.

In one embodiment, a device as described herein comprises a Li-ion battery.

In another embodiment, the device comprises a transparent cover. The transparent cover can be made of plastic and/or rubber, or high-density polyethylene (HDPE).

In one embodiment, the device as described herein can be a wristband, an ankle band, a lid or a cap configured to be removably attached to a container or a bottle, and/or any device (such as body strap bands) that can be worn by a human or animal subject for treatment purposes.

In one preferred embodiment, the device disclosed herein is an ankle band. In another preferred embodiment, the device disclosed herein is a lid or a cap configured to be removably attached to a container or a bottle. In yet another preferred embodiment, the device disclosed herein is a wristband.

In one aspect of a preferred embodiment, the ankle band or the wristband comprises a cupping. The cupping is specifically designed to (a) cup the area to be exposed for the at least 1 LED light to at least 10 LED lights and (b) to provide about 1/16 parts of an inch to about 1 inch from the skin to the LED lights. In one aspect described herein, the band comprises a silicone adjustable band.

One of skilled in the art would understand that the ankle band or the wristband as described herein can come in various combinations, including, for example, the wristband specifically exemplified in the drawings provided herein. As exemplified, the wristband of as described herein comprises as shown in FIGS. 3 and 4, a strap, a strap brand, and a button facing out for ease of wearability of the wristband.

In one aspect of a preferred embodiment, wherein the device is a lid or a cap configured to be removably attached to a container or a bottle, the light source comprised on the housing at the bottom of the cap or the lid (i.e., towards the side of the container or the bottle), is designed to be located at about 1/16 parts of an inch to about 4 inches from the a photosensitive compound or a composition comprising a photosensitive compound when placed in the container or bottle. In one embodiment, the lid or the cap comprises a transparent cover over the housing comprising the light source.

In one aspect of the preferred embodiment, wherein the device is a lid or a cap configured to be removably attached to a container or a bottle, the buttons may be located towards the outside of the cap on top or on the side of the cap. In yet one aspect of a preferred embodiment, wherein the device is a lid or a cap configured to be removably attached to a container or a bottle, the charging port may be located towards the outside of the cap on top or on the side of the cap.

In one embodiment, disclosed herein is a kit comprising the device. In one aspect of the embodiment disclosed herein, the kit can be a container. In another embodiment disclosed herein, the container comprises a light activation area for placing a photosensitive pharmaceutical ingredient or a drug product comprising a photosensitive ingredient. The photosensitive pharmaceutical ingredient, or the drug product can be a blister pack, bottle, a vial, an insert, a tablet, a capsule, or any form in which pharmaceutical ingredients/compositions are manufactured and/or stored or placed prior to administering to a subject.

In another embodiment, methods of activating a photosensitive drug are provided. In one aspect of the embodiment, the method of activating a photosensitive drug comprises (i) placing a photosensitive pharmaceutical ingredient or a drug product comprising a photosensitive ingredient in the light activation area of the kit described above, (ii) placing the device, for example the wristband, as described herein in the container as described, (iii) turning on the wristband to emit desired pattern of the at least 1 LED light to the at least 10 LED lights comprised within the housing, and (iv) activating the photosensitive pharmaceutical ingredient or the drug product comprising a photosensitive ingredient in the kit.

In one aspect of the embodiment, the method of activating a photosensitive drug comprises: (i) placing an intravenous (IV) bag in an IV bag comprising a photosensitive pharmaceutical ingredient or a drug product comprising a photosensitive ingredient in an IV bag cover as described herein, (ii) turning on the at least 1 LED light to the at least 10 LED lights comprised within the housing inside the IV bag, and (iii) activating the photosensitive pharmaceutical ingredient or the drug product comprising a photosensitive ingredient in the IV bag.

In another aspect of the embodiment, the method of activating a photosensitive drug comprises: (i) placing a photosensitive pharmaceutical ingredient or a drug product comprising a photosensitive ingredient in bottle, (ii) connecting the cap comprising the light source as described herein, (iii) turning on at least 1 LED light to the at least 10 LED lights comprised towards the inside the cap of the bottle, and (iv) activating the photosensitive pharmaceutical ingredient or the drug product comprising a photosensitive ingredient in the bottle.

In another embodiment, methods for treating a subject comprising exposing the skin or tissue to light emitted from the device disclosed herein. In one aspect of the embodiment, the method of treating a subject comprises administering to the subject a composition comprising a photosensitive compound. In one aspect of the embodiment, the method of treating a subject comprises administering to the subject a composition comprising a photosensitive compound prior to exposing the skin or tissue of the subject to the light emitted from the device disclosed herein. In another aspect of the embodiment, the method of treating a subject comprises administering to the subject a composition comprising a photosensitive compound after to exposing the skin or tissue of the subject to the light emitted from the device disclosed herein. In yet another aspect of the embodiment, the method of treating a subject comprises administering to the subject a composition comprising a photosensitive compound at the same time as exposing the skin or tissue of the subject to the light emitted from the device disclosed herein.

In one aspect of the embodiment, the method of treatment utilizing sublingual dosage of 0.1 ml to 5 ml held in the mouth for periods of time ranging from 30 seconds to 15 minutes described herein includes a treatment time (i.e., exposing the skin or tissue to light emitted from the device disclosed herein) that may range from about 30 seconds to about 12 minutes. In another aspect of the embodiment, the method of treatment described herein includes a treatment time of about 30 seconds to about 25 minutes, about 30 seconds to 20 minutes, about 30 seconds to 19 minutes, about 30 seconds to 18 minutes, about 30 seconds to 19 minutes, about 30 seconds to 17 minutes, about 30 seconds to 16 minutes, about 30 seconds to 15 minutes, about 30 seconds to 14 minutes, about 30 seconds to 13 minutes, about 30 seconds to 12 minutes, about 30 seconds to 1 minutes, about 30 seconds to 10 minutes, about 30 seconds to 9 minutes, about 30 seconds to 8 minutes, about 30 seconds to 7 minutes, about 30 seconds to 6 minutes, about 30 seconds to 5 minutes, about 30 seconds to 4 minutes, typically 5 to 15 minutes. The treatment time may range up to about 120 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area or to a subject.

In one aspect of the embodiment, there is provided a method of treating a subject infected with a virus comprising exposing the skin, tissue, blood, or plasma to light emitted from the device disclosed herein. In one aspect, the virus is an enveloped virus selected from, but not limited to, the group consisting of herpesviruses, poxviruses, hepadnaviruses, flavivirus, togavirus, coronavirus, hepatitis D, orthomyxovirus, rhabdovirus, bunyavirus, filovirus, retroviruses, and hepadnavirus, in addition to lime disease and acne.

In one aspect of another embodiment, the method of treating a subject comprises two wristbands as described herein. The wristbands can be worn on two hands, each wristband comprising anywhere from at least 1 LED light to at least 10 LED lights, wherein at least 1 LED light to at least 10 LED lights can be of same or different wavelengths. In one aspect of the embodiment described herein, the treatment comprises the wrist bands to be turned on at the same time. In another aspect of the embodiment described herein, the treatment comprises the wrist bands to be turned on in the alternative.

In one aspect of yet another embodiment, the method of treatment comprises a subject wearing a wrist band, wherein the subject is receiving an intravenous composition and wherein the wrist band is worn over intravenous tubing through which the composition flows through into the subject's body.

In one aspect of yet another embodiment, a device comprising at least 1 LED light to at least 10 LED lights can be configured to be worn on an intravenous (IV) bag is described herein. In one aspect of the embodiment, the device is a cover for an intravenous (IV) bag. One aspect of the embodiment comprises a method of activating a photosensitive drug in an IV bag, wherein the method comprises illuminating the IV bag by exposing the IV bag to a device as described herein.

In one aspect of yet another embodiment, a device comprising at least 1 LED light to at least 10 LED lights can be configured to illuminate the dosage form prior to administration as described herein.

In one aspect of yet another embodiment, a device comprising at least 1 LED light to at least 10 LED lights as described herein is a lid or a cap. The lid or the cap can be configured to be removably attached to a container or a bottle, wherein the container or the bottle can be used to place or store a photosensitive drug and/or a composition comprising a photosensitive drug.

In one aspect of the embodiment, method of activating a photosensitive drug, and/or a composition comprising a photosensitive drug, is described herein, wherein the method comprises placing the photosensitive drug and/or the composition comprising a photosensitive drug in the container or the bottle and attaching the lid or the cap to container or the bottle. In another aspect, the method further comprises activating the at least 1 LED light to at least 10 LED lights.

In some embodiments, the number of LED lights can be selected from the group consisting of 2 LED lights having a wavelength of 605 nm and 2 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm. In another embodiment, the number of LED lights can be selected from the group consisting of 3 LED lights having a wavelength of 605 nm and 3 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm. In another embodiment, the number of LED lights can be selected from the group consisting of 4 LED lights having a wavelength of 605 nm and 4 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm. In another embodiment, the number of LED lights can be selected from the group consisting of 5 LED lights having a wavelength of 605 nm and 5 LED lights having a wavelength of one or more selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm.

In some embodiments, the device comprises any number of at least 1 LED light to at least 10 LED lights having wavelength in various combinations.

In some embodiments, the device as described herein can comprise at least 1 LED light, at least 2 LED lights, at least 3 LED lights, at least 4 LED lights, at least 5 LED lights, at least 6 LED lights, at least 7 LED lights, at least 7 LED lights, at least 9 LED lights, and/or at least 10 LED lights, wherein the LED lights comprise the same and/or different wavelength ranging from about 600 nm to 705 nm.

For example, in some embodiments, the number of LED lights can be selected from the group consisting of 1 LED light, 2 LED lights, 3 LED lights, 4 LED lights, 5 LED lights, 6 LED lights, 7L ED lights, 8 LED lights, 9 LED lights, and 10 LED lights, wherein the LED lights have a wavelength selected from the group consisting of 600 nm, 610 nm, 615 nm, 620 nm, 625 nm, 630 nm, 635 nm, 640 nm, 645 nm, 650 nm, 655 nm, 660 nm, 665 nm, 670 nm, 680 nm, 685 nm, 690 nm, 695 nm, 700 nm, 705 nm, 710 nm, 715 nm, 720 nm, 725 nm, 730 nm, 735 nm, 740 nm, 745 nm, and 750 nm.

In one aspect of an embodiment described herein, the at least 1 LED light to the at least 10 LED lights carry a lighting power of using about 150 mA (milliamps) to about 3000 mA (milliamps) of lighting power and controlled watts. In yet another aspect of the embodiment the at least 1 LED light to the at least 10 LED lights carry a lighting power of using about 150 mA (milliamps). In yet another aspect of the embodiment the at least 1 LED light to the at least 10 LED lights carry a lighting power of using about 175 mA (milliamps). In yet another aspect of the embodiment the at least 1 LED light to the at least 10 LED lights carry a lighting power of using about 200 mA (milliamps). In yet another aspect of the embodiment the at least 1 LED light to the at least 10 LED lights carry a lighting power of using about 225 mA (milliamps). In yet another aspect of the embodiment the at least 1 LED light to the at least 10 LED lights carry a lighting power of using about 250 mA (milliamps). In yet another aspect of the embodiment the at least 1 LED light to the at least 10 LED lights carry a lighting power of using about 275 mA (milliamps). In yet another aspect of the embodiment the at least 1 LED light to the at least 10 LED lights carry a lighting power of using about 300 mA (milliamps). In yet another aspect of the embodiment the at least 1 LED light to the at least 10 LED lights carry a lighting power of using about 325 mA (milliamps). In yet another aspect of the embodiment the at least 1 LED light to the at least 10 LED lights carry a lighting power of using about 350 mA (milliamps). In yet another aspect of the embodiment the at least 1 LED light to the at least 10 LED lights carry a lighting power of using about 375 mA (milliamps). In yet another aspect of the embodiment the at least 1 LED light to the at least 10 LED lights carry a lighting power of using about 400 mA (milliamps). In yet another aspect of the embodiment the at least 1 LED light to the at least 10 LED lights carry a lighting power of using about 425 mA (milliamps). In yet another aspect of the embodiment the at least 1 LED light to the at least 10 LED lights carry a lighting power of using about 450 mA (milliamps). In yet another aspect of the embodiment the at least 1 LED light to the at least 10 LED lights carry a lighting power of using about 475 mA (milliamps). In yet another aspect of the embodiment the at least 1 LED light to the at least 10 LED lights carry a lighting power of using about 500 mA (milliamps). In yet another aspect of the embodiment the at least 1 LED light to the at least 10 LED lights carry a lighting power of using about 750 mA (milliamps). In yet another aspect of the embodiment the at least 1 LED light to the at least 10 LED lights carry a lighting power of using about 1000 mA (milliamps). In yet another aspect of the embodiment the at least 1 LED light to the at least 10 LED lights carry a lighting power of using about 1250 mA (milliamps). In yet another aspect of the embodiment the at least 1 LED light to the at least 10 LED lights carry a lighting power of using about 1500 mA (milliamps). In one aspect of an embodiment described herein, the at least 1 LED light to the at least 10 LED lights carry a lighting power of using about 150 mA (milliamps) to about 3000 mA (milliamps) of lighting power and controlled watts.

In one aspect of an embodiment, the at least 1 LED light to the at least 10 LED lights, in any combination described herein, can pass through the skin at depths of about 0.1 mm to about 30.0 mm. In yet another aspect of an embodiment, the at least 1 LED light to the at least 10 LED lights, in any combination described herein, can pass through the skin at depths selected from the group consisting of about 0.1 mm, about 0.25, about 0.5 mm, about 0.75 mm, about 1.0 mm, about 1.25 mm, about 1.5 mm, about 1.75 mm, about 2.0 mm, about 2.25 mm, about 2.5 mm, about 2.75 mm, about 3.0 mm, about 3.25 mm, about 3.5 mm, about 3.75 mm, about 4.0 mm, about 4.25 mm, about 4.5 mm, about 5.0 mm, about 6 mm, about 7 mm, about 8 mm, about 9 mm, about 10 mm, about 12.5 mm, about 15 mm, about 17.5 mm, about 20 mm, about 22.5 mm, about 25.0 mm, about 27.5 mm, and about 30.0 mm. In one aspect of an embodiment, the at least 1 LED light to the at least 10 LED lights, in any combination described herein, can pass through the skin at depths of about 0.1 mm to about 5.0 mm. in yet another aspect the at least 1 LED light to the at least 10 LED lights, in any combination described herein, can pass through the skin at depths selected from the group consisting of about 0.1 mm to about 4.0 mm, about 0.1 mm to about 3.5 mm, about 0.1 mm to about 3.0 mm, about 0.1 mm to about 2.5 mm, about 0.1 mm to about 2.0 mm, about 0.1 mm to about 6 mm, about 1 mm to about 5 mm, about 2.5 mm to about 7.5 mm, about 5 mm to about 10 mm, about 7.5 mm to about 15 mm, about 10 mm to about 20 mm, about 15 mm to about 20 mm, about 20 mm to about 25 mm and about 20 mm to about 30 mm.

In one aspect of an embodiment described herein, the light emitted from the light source generates a temperature between about 40° F. to about 100° F. In yet another aspect of an embodiment described herein, the light emitted from the light source generates a temperature selected from the group consisting of about 40° F., about 45° F., about 50° F., about 55° F., about 60° F., about 65° F., about 70° F., about 75° F., about 80° F., about 85° F., about 90° F., about 95° F., and about 100° F. In yet another aspect of an embodiment described herein, the light emitted from the light source generates a temperature selected from the group consisting of about 45° F. to about 95° F., about 50° F. to about 90° F., about 55° F. to about 85° F., about 60° F. to about 80° F., and about 65° F. to about 75° F.

In one aspect an embodiment, a device described herein (such as an ankle band or the wristband) comprises a cupping, wherein cupping provides for a distance of about 1/16 parts of an inch to about 1 inch from the skin to the LED lights. In yet another aspect of an embodiment described herein, the cupping provides for a distance of about 1/16 parts of an inch from the skin to the LED lights. In yet another aspect of an embodiment described herein, the cupping provides for a distance of about ⅛ parts of an inch from the skin to the LED lights. In yet another aspect of an embodiment described herein, the cupping provides for a distance of about ¼ parts of an inch from the skin to the LED lights. In yet another aspect of an embodiment described herein, the cupping provides for a distance of about ½ of an inch from the skin to the LED lights. In yet another aspect of an embodiment described herein, the cupping provides for a distance of about 1 of an inch from the skin to the LED lights.

A device disclosed in a preferred embodiment disclosed herein is designed to activate drug products and/or pharmaceutical active ingredients that are light sensitive to increase the therapeutic effect either when directly subjected to the light in a container or a finished drug product form acceptable to the United States Food and Drug Administration or while passing through the blood stream of the human or pet body after administration either by injection, orally or intra nasal application. The device is constructed to enable the active pharmaceutical ingredient to become more effective when administered to a human or pet.

One of the preferred embodiments disclosed herein is a device for photodynamic inactivation of envelope viruses. Photodynamic inactivation uses specific wavelengths of light to activate special compounds that leads to the formation of reactive oxygen species which can literally render certain envelope viruses powerless.

Many envelop viruses such as SARS, Malaria, AIDS and other envelope viruses are destroyed or greatly reduced when using this device to light activate drugs that are administered in conjunction with the support of specific light frequencies that passes through the skin, as designed in the device.

A device disclosed herein can specifically designed, but not limited to, with the proper sequencing of LED light arrangements, distance between the LED lights (both domed and non-domed) in the device, distance from the skin, Nanometer wavelength, time of illumination and illumination power focused in directional light beams in a skin area available to penetrate to the levels of therapeutic effect or increase of drug molecule activity, for the wristband lighting to be effective.

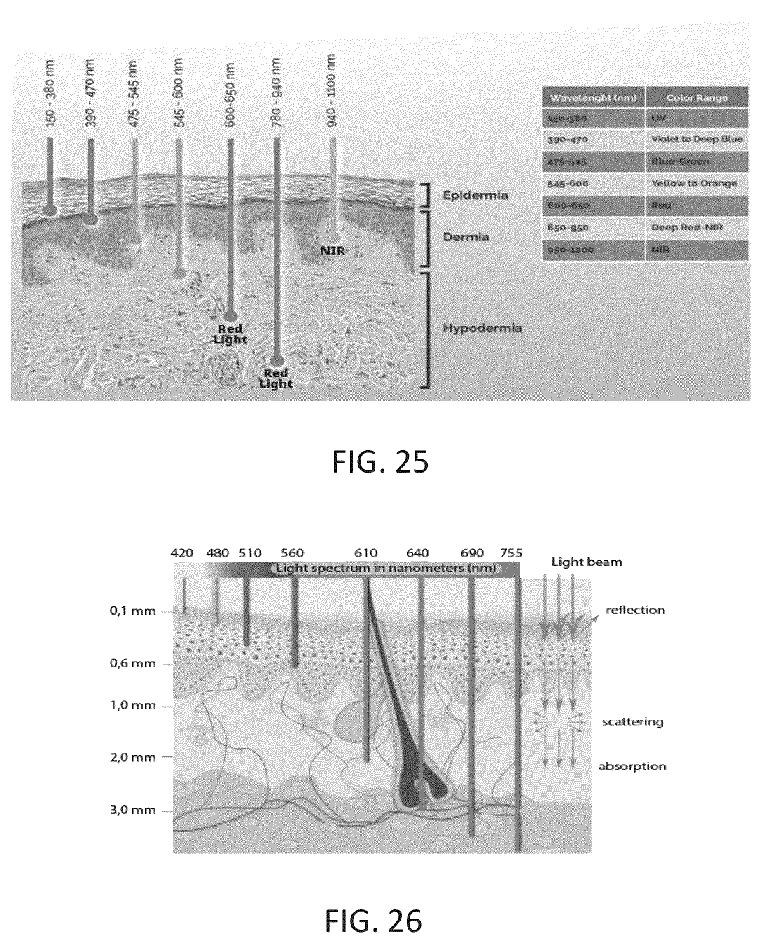

The wristband is constructed to provide this arrangement of LED lights along with an adjustable or fixed distance of the lighting illumination from the human or pet skin. FIGS. 25 and 26 provide a description of light frequency penetration.

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical and electrical changes can be made without departing from the scope of what is claimed. The following detailed description is, therefore, not to be taken in a limiting sense, and the scope is defined by the appended claims and their equivalents.

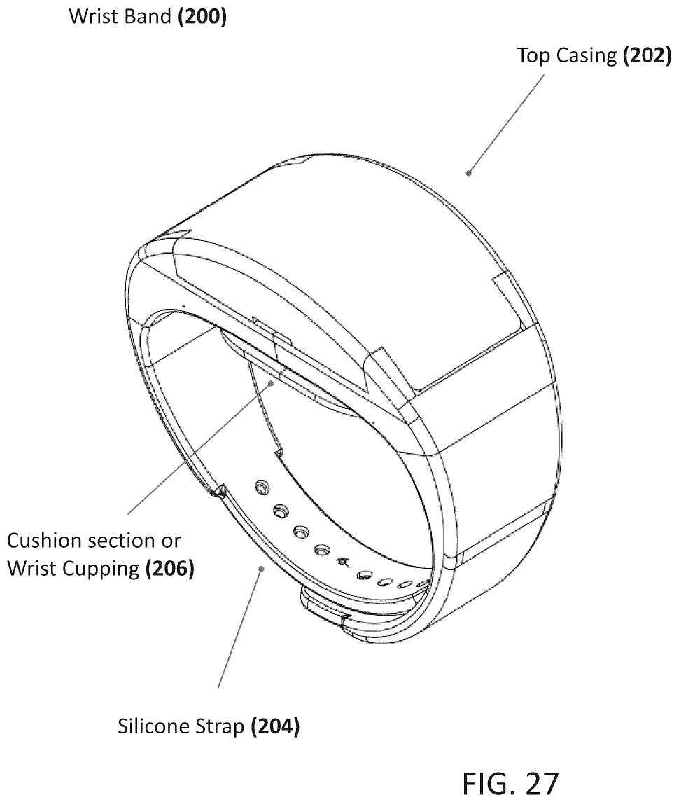

FIG. 27 depicts a phototherapeutic device, a wristband (200), in accordance with present disclosure. The wristband (200) comprises a top casing (202) and a silicone strap (204), with a cushion section or wrist cupping section (206).

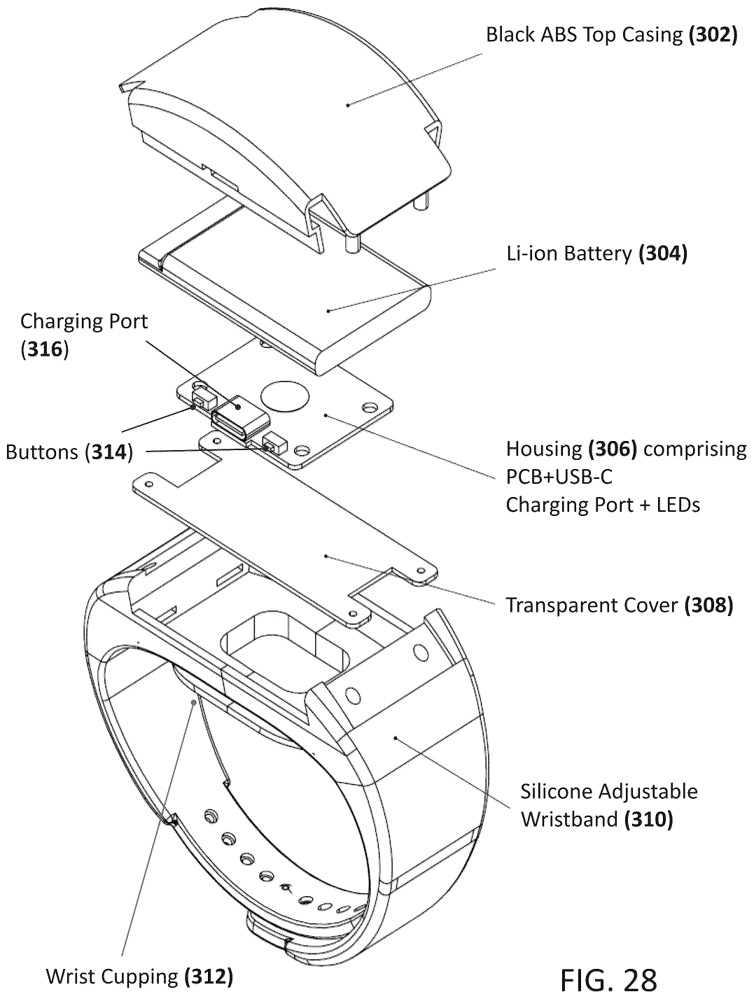

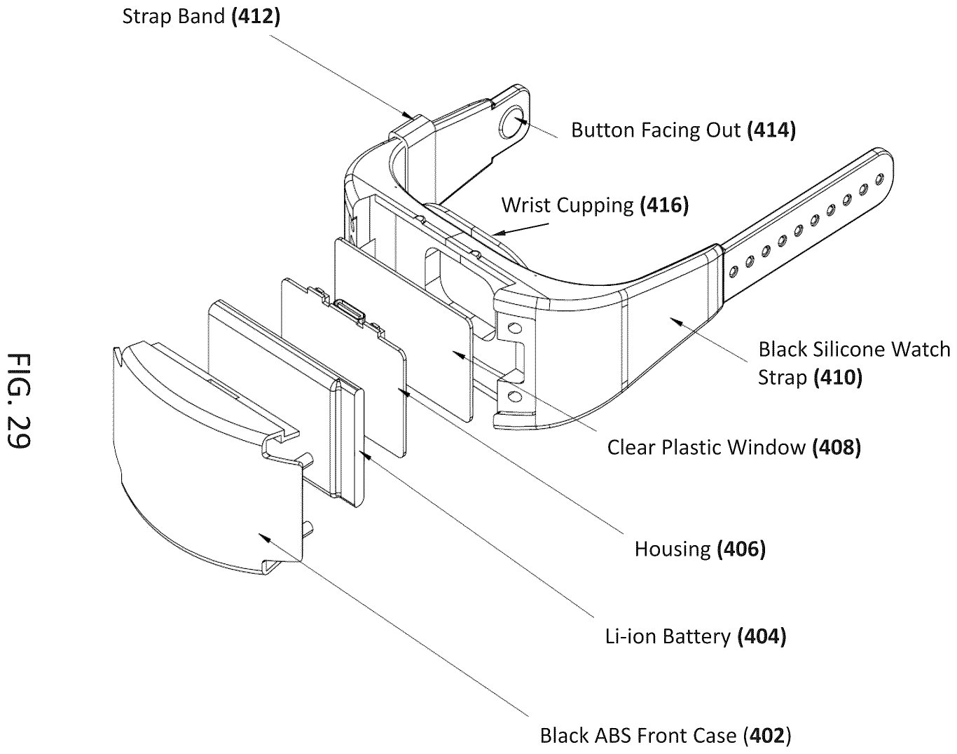

FIG. 28 depicts a phototherapeutic device in accordance with an embodiment of present disclosure. The device comprises a black ABS top casing (302), a Li-ion battery (304), a housing (306), transparent cover (308), and a silicone adjustable wristband (310) comprising a wrist cupping (312). The housing (306) comprises PCB+USB-C charging port (316), buttons (314), and LEDs.

FIG. 29 depicts a different view of the wristband as disclosed herein and shows the black ABS top casing (402), a Li-ion battery (404), housing (406), clear plastic window (408), and a silicone watch strap (410), a strap band (412), button facing out (414), and a wrist cupping (416).

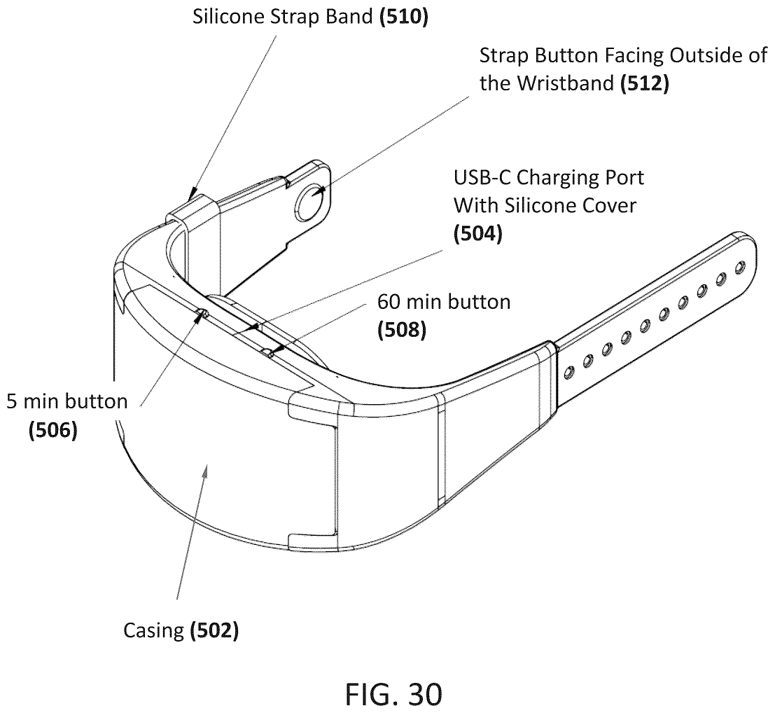

FIG. 30 depicts a different view of the wristband as disclosed herein and shows the casing (502), the housing comprising USB-C charging port with silicone cover (504), a 5-minute button, for example, (506), a 60-minute button, for example, (508), a silicone strap band (510), and a strap button facing outside of the wristband (512).

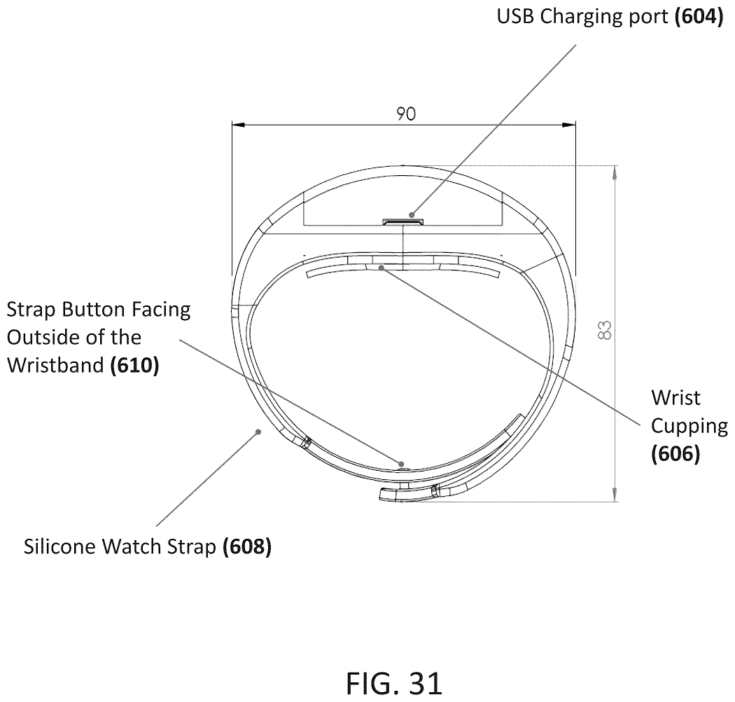

FIG. 31 depicts a different view of the wristband as disclosed herein and shows the USB-C charging port (604), wrist cupping (606), a silicone watch strap (608), and a strap button facing outside of the wristband (610).

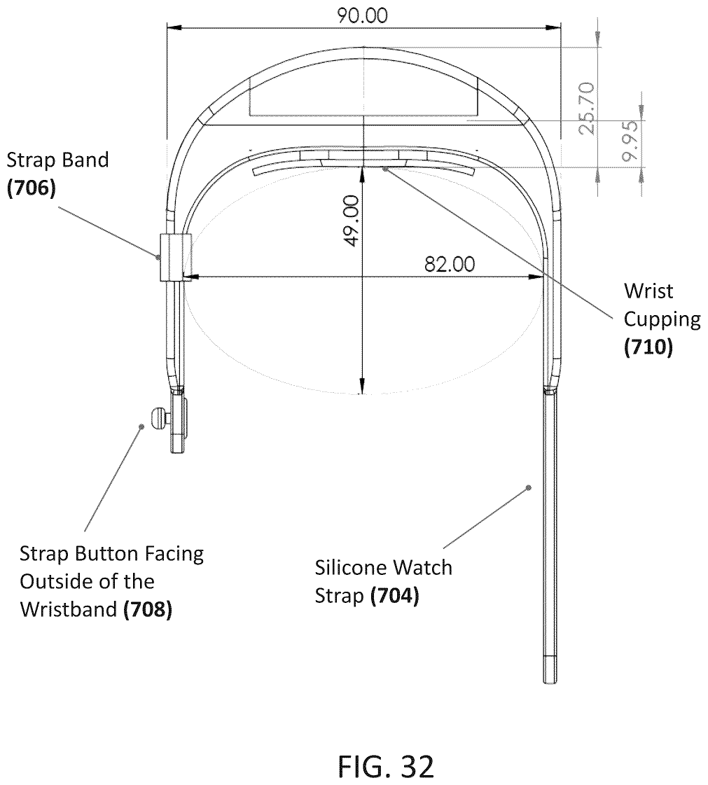

FIG. 32 depicts a different view of the wristband as disclosed herein and shows the silicone watch strap (704), strap band (706), strap button facing outside of the wristband (708), and a wrist cupping (710).

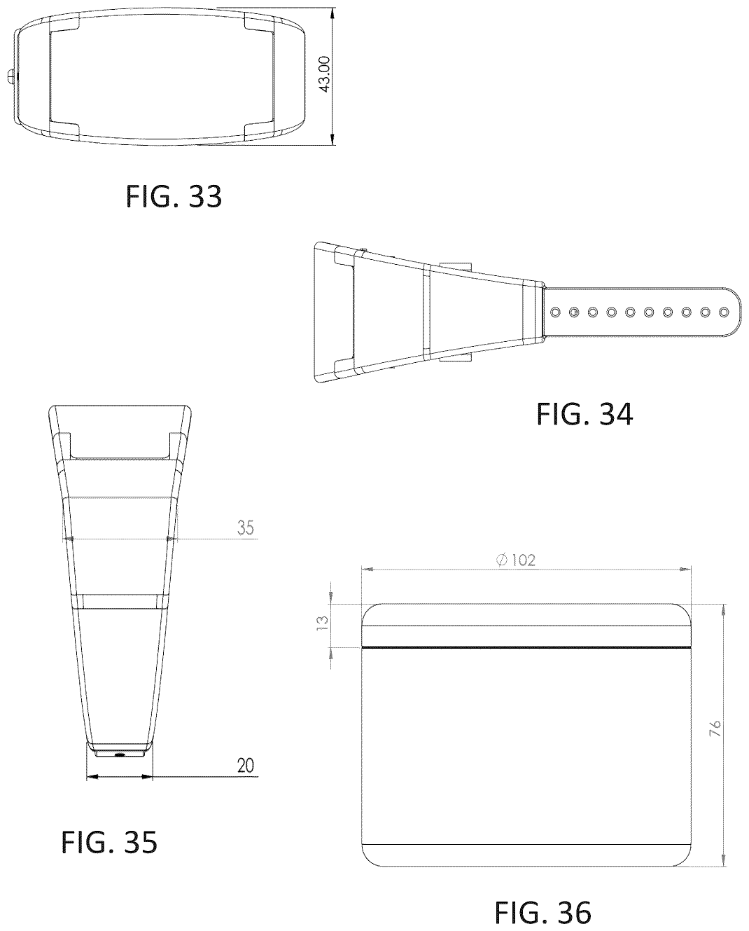

FIGS. 33, 34 and 35 further illustrate a different view of the wristband as disclosed herein.

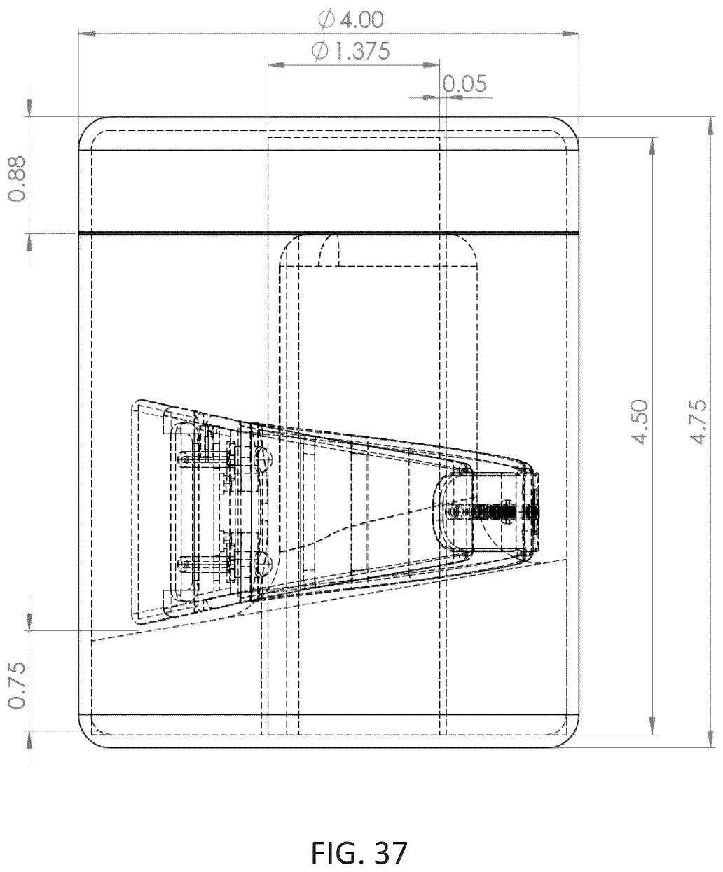

FIG. 36 depicts the outside view of a container as described herein.

FIG. 37 depicts a container (130) according to an embodiment described herein. The container comprises a base (132) which is adapted to fit the wristband (136) in such a way that the wrist cupping (136) of the wristband is facing towards the inside of the container. The container base (132) is also adapted to have a light activation area (138). The light activation area (138) comprises a structure around which the wrist band (136) can be placed, wherein the structure comprises (i) space within to place, for example a bottle comprising, a photosensitive drug or a composition comprising the photosensitive drug, and (ii) space, as wide as the wristband cupping, on the side.

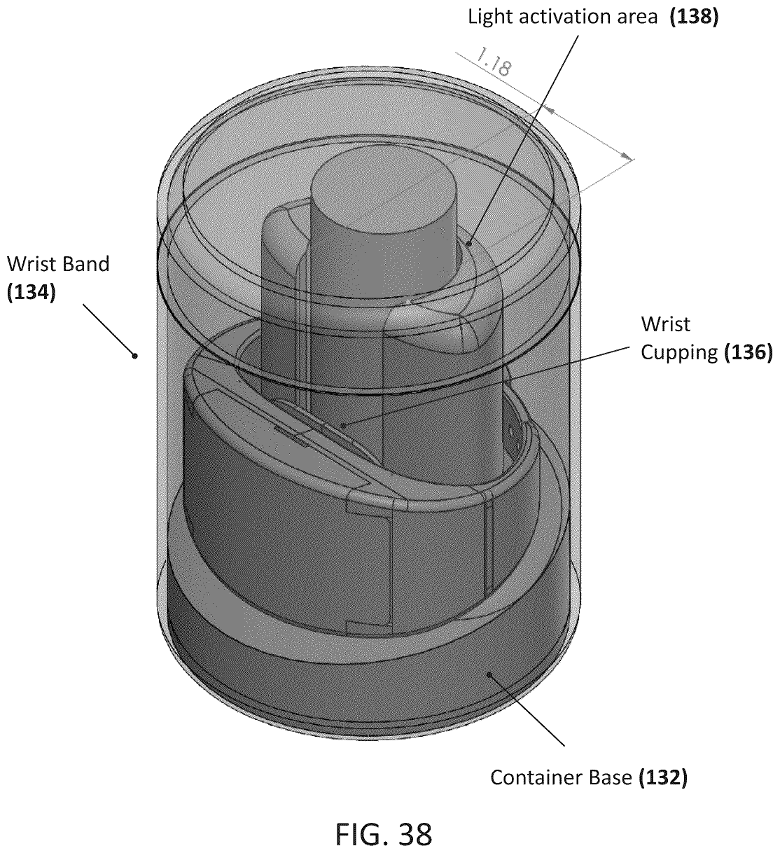

FIG. 38 depicts the way the wristband may be placed in a container. It shows a wrist band placed in a container in such a manner that the light from the at least 1 LED light to the at least 10 LED lights is perpendicular to the unit within the light activation area (138). sleeve (138) allows for placing of a unit comprising a photosensitive pharmaceutical ingredient or a drug product. One of skilled in the art can readily imagine that although FIG. 38 shows the container with a sleeve (138), embodiments of the container without such sleeve are also contemplated. Such container without sleeves would allow for the unit comprising a photosensitive pharmaceutical ingredient or a drug product to be placed within the area of the wrist strap of the wristband when placed in the container.

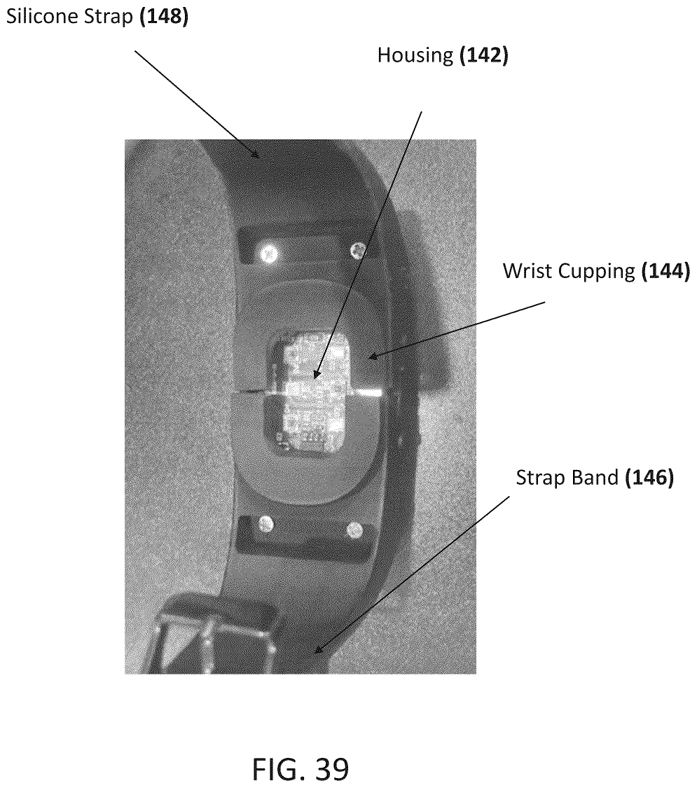

FIG. 39 depicts the bottom view of a wristband as described herein and shows the backside of the housing (142) from within the wristband, wrist cupping (144), strap band (146), and the silicone strap (148).

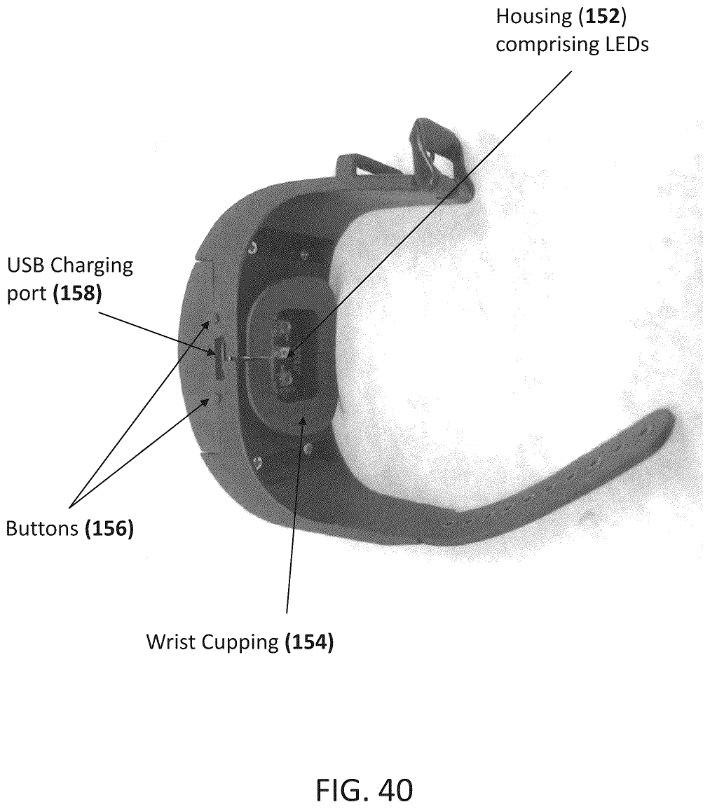

FIG. 40 depicts a different view of the wristband as disclosed herein and shows the housing (152) comprising LEDs, wrist cupping (154), buttons (156), and the USB charging port (158).

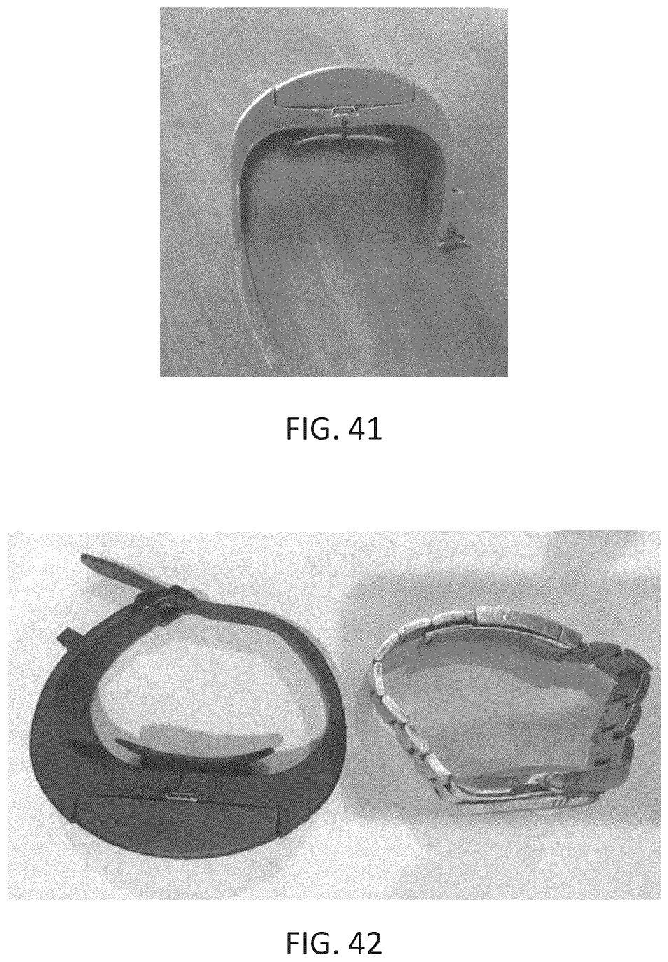

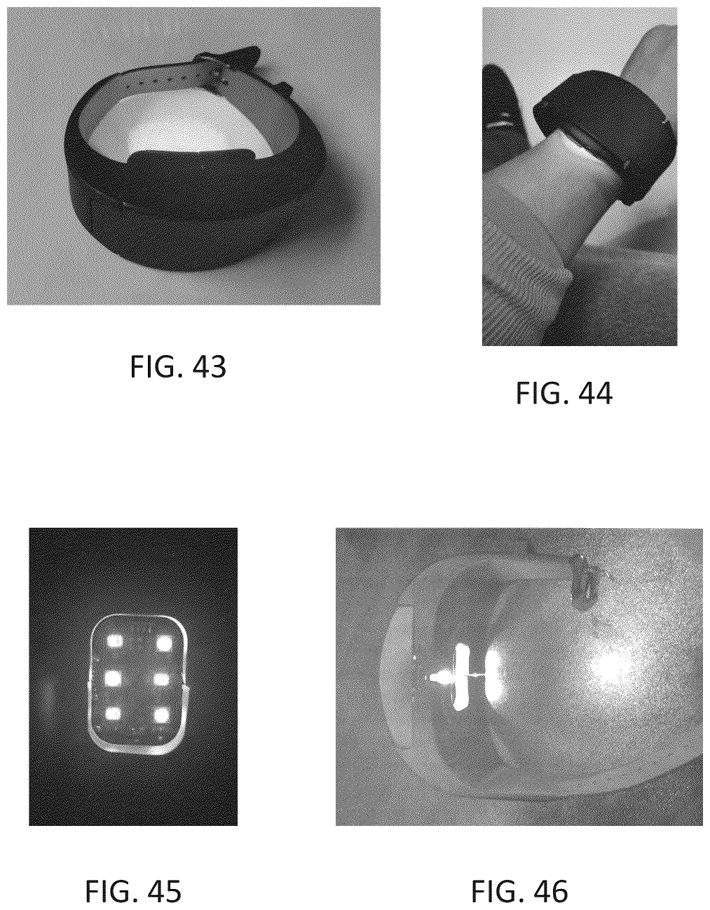

FIG. 41 depicts the top view of the wristband described herein, wherein the wristband is rested on its side. FIG. 42 depicts the size of the wristband described herein with a side-by-side comparison of the wristband with a hand watch. FIG. 43 depicts the wristband as described herein, wherein the wristband is illuminated. FIG. 44 depicts a subject wearing the wristband as described herein. FIG. 45 depicts the bottom view of the housing component of the wristband, wherein the LED lights in the housing component as illuminated. FIG. 46 depicts the wristband as described herein rested on its side, wherein the wristband is illuminated.

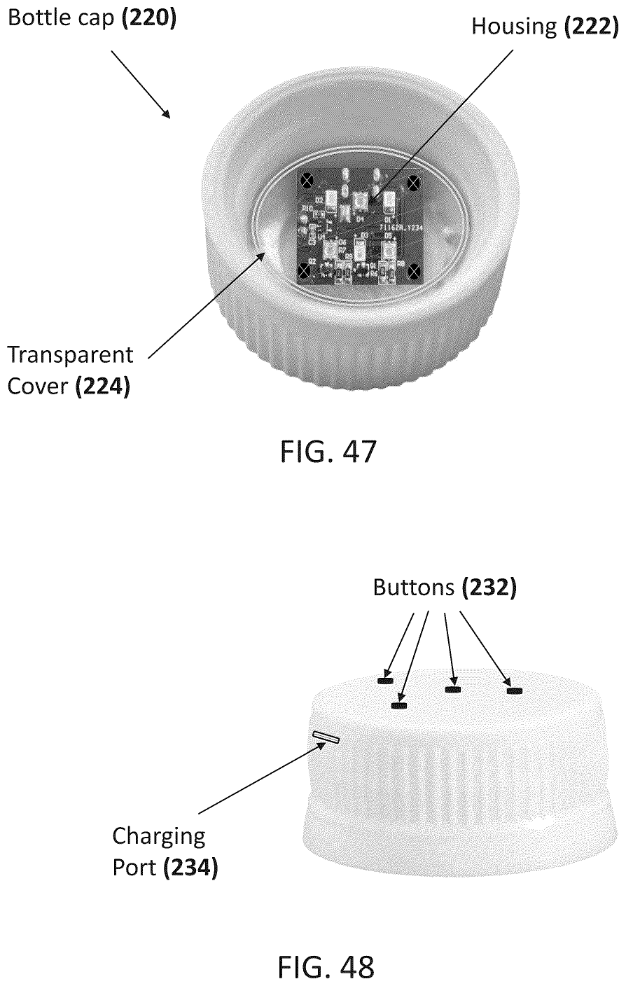

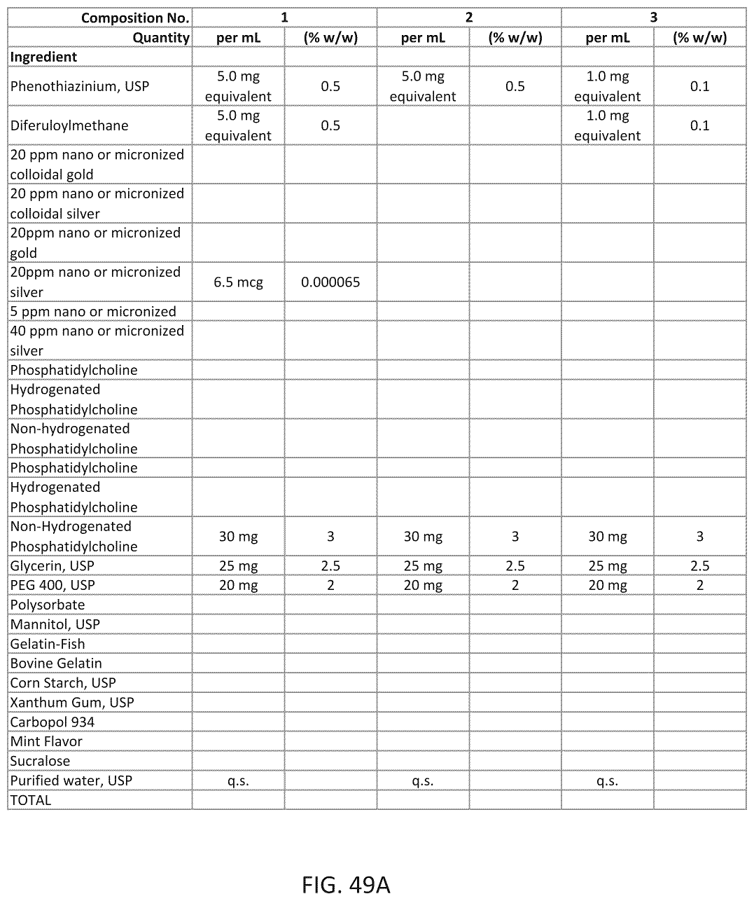

FIG. 47 depicts the inside view of a bottle cap (220) according to an embodiment described herein. The bottle cap (220) comprises a housing (222) comprising the light source as described herein, and a transparent cover (224) over the housing (222). FIG. 48 depicts the outside of the bottle cap comprising buttons (232) and a charging port (234). One or ordinary skill in the art would clearly understand that a cap can be designed with different combination of the placement of the buttons and the charging port that differ from that depicted in FIG. 48.

In one embodiment disclosed herein, methods of treating subjects diagnosed with bacterial or viral infections as well as preventing the onset of bacterial or viral infections comprising administering a pharmaceutically effective amount of a composition described herein to a subject in need thereof are provided.

In one embodiment disclosed herein, the method of treating or preventing a subject diagnosed with bacterial and/or viral infections as well as preventing the onset of bacterial and/or viral infections comprises administering to a subject need thereof the composition described herein, wherein the composition is not illuminated.

In another embodiment disclosed herein, the method of treating or preventing a subject diagnosed with bacterial and/or viral infections as well as preventing the onset of bacterial and/or viral infections comprises administering to a subject need thereof the composition described herein, wherein the composition is illuminated.

In one embodiment disclosed herein, the method of treating or preventing a subject diagnosed with bacterial and/or viral infections as well as preventing the onset of bacterial and/or viral infections comprises administering to a subject need there of the solid composition described herein.

In yet another embodiment disclosed herein, the method of treating or preventing a subject diagnosed with bacterial and/or viral infections as well as preventing the onset of bacterial and/or viral infections comprises administering to a subject need thereof the liquid composition described herein.

In yet another embodiment disclosed herein, the method of treating or preventing a subject diagnosed with bacterial and/or viral infections as well as preventing the onset of bacterial and/or viral infections comprises administering to a subject need thereof the solid composition described herein.

In yet another embodiment disclosed herein, the method of treating or preventing a subject diagnosed with bacterial and/or viral infections as well as preventing the onset of bacterial and/or viral infections comprises administering to a subject need thereof the composition intranasally. In an aspect of the embodiment described herein, the method of administering to a subject need thereof the composition intranasally comprises a nebulizer.

The present disclosure provides methods for activating a photosensitive compound. In one aspect, the method comprises placing the photosensitive compound or a composition comprising such photosensitive compound in the container disclosed herein and further exposing the compound or the composition to the lights emitted by a device described herein. In one aspect the step of exposing may last for from about 30 seconds to about 20 minutes. In yet another aspect the step of exposing may last for a time selected from the group consisting of about 30 seconds, about 40 seconds, about 50 seconds, about 60 seconds, about 70 seconds, about 80 seconds, and about 90 minutes. In yet another aspect the step of exposing may last for a time selected from the group consisting of about 30 seconds, about 40 seconds, about 50 seconds, about 60 seconds, about 70 seconds, about 80 seconds, about 90 seconds, about 2 minutes, about 3 minutes, about 4 minutes, about 5 minutes, about 6 minutes, about 7 minutes, about 8 minutes, about 9 minutes, about 10 minutes, about 11 minutes, and about 12 minutes. In yet another aspect the step of exposing may last for a time selected from the group consisting of about 30 seconds to about 45 seconds, 30 seconds to about 90 seconds, 60 seconds to about 90 seconds, about 1 minute to about 2 minutes, about 1 minute to about 3 minutes, about 1 minute to about 4 minutes, about 2 minutes to about 3 minutes, about 1 minute about 3 minutes, about 2 minutes to about 5 minutes, about 5 minutes to about 7.5 minutes, about 7.5 minutes to about 10 minutes, and about 10 minutes to about 20 minutes.

In another embodiment, methods of activating a photosensitive drug are provided. In one aspect of the embodiment, the method of activating a photosensitive drug comprises (i) placing a photosensitive pharmaceutical ingredient or a drug product comprising a photosensitive ingredient in the light activation area of the kit described above, (ii) placing the device, for example the wristband, as described herein in the container as described, (iii) turning on the wristband to emit desired pattern of the at least 1 LED light to the at least 10 LED lights comprised within the housing, and (iv) activating the photosensitive pharmaceutical ingredient or the drug product comprising a photosensitive ingredient in the kit.

In one aspect of the embodiment, the method of activating a photosensitive drug comprises: (i) placing an intravenous (IV) bag or tubing in an IV bag or tubing comprising a photosensitive pharmaceutical ingredient or a drug product comprising a photosensitive ingredient in an IV bag cover as described herein, (ii) turning on the at least 1 LED light to the at least 10 LED lights comprised within the housing inside the IV bag, and (iii) activating the photosensitive pharmaceutical ingredient or the drug product comprising a photosensitive ingredient in the IV bag.

In one aspect of the embodiment, the method of activating a photosensitive drug in the blood stream comprises: (i) placing the device on the pulse side of the wrist or any other accessible pules location of the body, (ii) turning on the at least 1 LED light to the at least 10 LED lights comprised within the device (iii) device turned on from 1 to 120 minutes. (iv) activating the photosensitive pharmaceutical ingredient or the drug product comprising a photosensitive ingredient in the blood stream.

In one aspect of the embodiment, the method of activating a photosensitive drug comprises: (i) placing a tablet, capsule or liquid or mist comprising a photosensitive pharmaceutical ingredient or a drug product comprising a photosensitive ingredient as described herein, (ii) turning on the device comprising the at least 1 LED light to the at least 10 LED lights comprised directed at tablet, capsule or liquid or mist and (iii) activating the photosensitive pharmaceutical ingredient or the drug product comprising a photosensitive ingredient in the IV bag.

In another aspect of the embodiment, the method of activating a photosensitive drug comprises: (i) placing a photosensitive pharmaceutical ingredient or a drug product comprising a photosensitive ingredient in bottle, (ii) connecting the cap comprising the light source as described herein, (iii) turning on at least 1 LED light to the at least 10 LED lights comprised towards the inside the cap of the bottle, and (iv) activating the photosensitive pharmaceutical ingredient or the drug product comprising a photosensitive ingredient in the bottle.

The present disclosure provides methods of treating a subject comprising exposing the skin or tissue to light emitted from the device disclosed herein. In one aspect of the embodiment, the method of treating a subject comprises administering to the subject a composition comprising a photosensitive compound. In one aspect of the embodiment, the method of treating a subject comprises administering to the subject a composition comprising a photosensitive compound prior to exposing the skin or tissue of the subject to the light emitted from the device disclosed herein. In one aspect of the embodiment, the method of treating a subject comprises administering to the subject a composition comprising a photosensitive compound after to exposing the skin or tissue of the subject to the light emitted from the device disclosed herein. In one aspect of the embodiment, the method of treating a subject comprises administering to the subject a composition comprising a photosensitive compound at the same time as exposing the skin or tissue of the subject to the light emitted from the device disclosed herein.

The present disclosure also provides methods of treating subjects diagnosed with bacterial and/or viral infections as well as preventing the onset of bacterial and/or viral infections. In one embodiment disclosed herein, methods for the treatment of bacterial- and/or viral-related symptoms are provided. In another embodiment disclosed herein, methods for the prevention or delay in development of bacterial- and/or viral-related complications are provided.

In one aspect of an embodiment disclosed herein, the bacteria belong to the family Staphylococcaceae. In one aspect of an embodiment disclosed herein, the bacteria is selected from the group consisting of S. argenteus, S. aureus, S. schweitzeri, S. simiae, S. auricularis, S. carnosus, S. condimenti, S. debuckii, S. massiliensis, S. piscifermentans, S. simulans, S. capitis, S. caprae, S. epidermidis, S. saccharolyticus, S. borealis, S. devriesei, S. haemolyticus, S. hominis, S. agnetis, S. chromogenes, S. cornubiensis, S. felis, S. delphini, S. hyicus, S. intermedius, S. lutrae, S. microti, S. muscae, S. pseudintermedius, S. rostri, S. schleiferi, S. lugdunensis, S. arlettae, S. caeli, S. cohnii, S. equorum, S. gallinarum, S. kloosii, S. leei, S. nepalensis, S. saprophyticus, S. succinus, S. xylosus, S. sciuri group-S. fleurettii, S. lentus, S. sciuri, S. stepanovicii, S. vitulinus, S. simulans, S. pasteuri, S. warneri, and S. caseolyticus. In another aspect of an embodiment disclosed herein, the bacteria is selected from the group consisting of S. aureus subsp. Aureus, S. aureus subsp. Anaerobius, S. capitis subsp. Capitis, S. capitis subsp. Urealyticus, S. carnosus subsp. Carnosus, S. carnosus subsp. Utilis, S. cohnii subsp. Cohnii, S. cohnii subsp. Urealyticus, S. equorum subsp. Equorum, S. equorum subsp. Linens, S. hominis subsp. Hominis, S. ominis subsp. Novobiosepticus, S petrasii subsp. Croceilyticus, S petrasii subsp. Jettensis, S petrasii subsp. Petrasii, Spetrasii subsp. Pragensis, S. saprophyticus subsp. Bovis, S. saprophyticus subsp. Saprophyticus, S. schleiferi subsp. Coagulans, S. schleiferi subsp. Schleiferi, S. sciuri subsp. Carnaticus, S. sciuri subsp. Rodentium, S. sciuri subsp. Sciuri, S. succinus subsp. Casei, and S. succinus subsp. Succinus.

In one aspect of an embodiment disclosed herein, the virus can be selected from the group consisting of herpes simplex virus 1, herpes simplex virus 2, herpes zoster (shingles), human herpes virus 1, human virus 2, human herpes virus 3, Epstein-Barr virus or human herpes virus 4, human cytomegalovirus or human herpes virus 5, human herpes virus 6A, human herpes virus 6B, human herpes virus 7, Kaposi sarcoma-associated herpes virus or human herpes virus 8, smallpox virus, vaccinia virus, cowpox virus, monkeypox virus, rabbitpox virus, orf virus, pseudocowpox, bovine papular stomatitis virus, tanapox virus, yaba monkey tumor virus, molluscum contagiosum virus, hepatitis virus B, African swine fever virus, west nile virus, dengue virus, tick-borne encephalitis virus, yellow fever virus, zika virus, palm creek virus, parramatta river virus, yellow fever virus, alphavirus, togavirus, coronaviruses, hepatitis A virus, hepatitis B virus, hepatitis C virus, hepatitis D virus, influenza virus, alphainfluenzavirus, betainfluenzavirus, deltainfluenzavirus, gammainfluenzavirus, isavirus, thogotovirus, and quaranjavirus, paramoxyvirus, rhabdovirus, bunyavirus, human immunodeficiency virus-1 (HIV-1), human immunodeficiency virus-2 (HIV-2), and human T-lymphotropic virus (HTLV).

FIG. 1 is an illustration of the cellular mechanism of action of photosensitizer, according to embodiments herein. In the present study, zwitterionic polymer-lipid was self-assembled into liposomes and encapsulate methylene blue (MB-liposome). Its properties of high stability and fast intracellular uptake were confirmed, and the higher in vitro ROS generation ability of MB-liposomes than that of free methylene blue (MB) was also verified. With the features found, MB-liposome has the potential of being an effective PDT Nano agent for antimicrobial therapy.

FIG. 2 depicts stepwise photo inactivation of multidrug resistant microbes by monomeric MB conjugated gold nanoparticles.

FIG. 3 depicts the monomeric Phenothiazinium category molecule on Gold nano particles. It enhances the fluorescence decay time and O2.

FIG. 4 shows images of TEM, SEM, and Confocal Laser Scanning Microscopy (CLSM) analysis showing photo-cytotoxicity. ROS probes and PCA analysis showed the cytoplasmic 102 is antimicrobial agent. The Methylene Blue Gold Nanoparticle didn't show any photo-cytotoxicity against mammalian cells.

FIG. 5(a) depicts TEM image of Methylene blue-liposomes and 5(b) is a graph showing Dynamic light scattering (DLS) measurement of MB-liposomes.

FIG. 6(a) depicts graph showing UV-Vis's spectra of free MB, MB-liposomes, and MB-liposomes with Nano Gold & silver 4.5 mcg & 6.5 mcg respectively, and 6(b) depicts graph showing stability of present composition: hydrodynamic diameter of MB-liposomes over 14 days.

FIG. 7(a) depicts TEM images of Phenothiazinium category molecule while 7(b) depicts graph showing particle size distribution of 60 nm to 140 nm and its intensity of absorbance.

FIGS. 8(a) to 8(e) depicts Phenothiazinium category molecule Liposomal life cycle; wherein FIG. 8(a) Phenothiazinium category molecule liposomal entrapped; FIG. 8(b) Phenothiazinium category molecule liposomal coating removal; FIG. 8(c) Phenothiazinium category molecule liposomal coating removal and treated human like enzyme are images; FIG. 8(d) Particle size distribution; and FIG. 8(e) zeta potential of various are graphs showing Phenothiazinium category molecule size and zeta potential;

FIG. 9(a) depicts graph showing 24-h cell viability (by MTT assay) of free MB and MB-liposomes and Distilled Water (DI) as control at different concentrations with dark treatment and photodynamic therapy (PDT) treatment.

FIG. 9(b) depicts images of the free MB and MB-liposomes at 8 μm concentration and Distilled Water (DI) with dark treatment and photodynamic therapy (PDT) treatment.

FIG. 10 is a schematic representation depicting synthesis of silver nano particles with Liposomal MB.

FIG. 11(a) depicts SEM image of Curcumin nanoparticles.

FIG. 11(b) depicts SEM image of silver & gold nanoparticles.

FIG. 12(a), FIG. 12(b) and FIG. 12(c) depicts SEM images of colloidal gold nanorods.

FIG. 13 depicts Photodynamic Diagnosis oxidation rate and its microscopic images; wherein 13(a) is an image of Phenothiazinium category molecule 13(b) is an image of Phenothiazinium category molecule liposomal form 13(c) is a graph showing various rate of oxidation rate impact by various wavelength and 13(d) is an image of Phenothiazinium category molecule liposomal form sonification.

FIG. 14 (a) depicts blue light excitation of Liposomal-MB while 14(b) depicts red light excitation of Liposomal-MB. Under blue light, Liposomal-MB is excited and emits red fluorescence. Its detection can guide the surgical resection of the fluorescent tissues. By exciting MB with red light, reactive oxygen species such as singlet O2 are formed through photochemical reactions and energy transfer. These cause oxidative damage to vital cellular components such as lipids, proteins and nucleic acids, culminating in massive tumor cell death.

FIG. 15 depicts schematic representation of (Peritoneal carcinomatosis) PCAR treatment using PDT. Promising results from the application of chemotherapy for PCAR have been achieved using innovative photo immunoconjugates and photosensitizer-loaded nanocarriers, which can in principle be combined to develop targeted photo nanomedicines. Regarding the integration into the clinical practice, PDT may synergize with clinical chemotherapies, and innovative lighting solutions (e.g., implantable light panels and diffusing tip laser wands) may aid in improving the efficacy of PDT for PCAR. To overcome the difficulty of reaching occult PCAR with light, radiotherapy.

FIG. 16 depicts schematic overview of the use of radio therapy activated PDT for PCAR using radio luminescent nanomaterials.

FIG. 17 depicts a schematic representation of photodynamic production of cytotoxic reactive oxygen species by nanoparticles. Scintillating nanomaterials accumulated in tissues can absorb the X-ray radiation and down-convert it into visible light. Conjugated photosensitizers can be excited, resulting in the photodynamic production of cytotoxic reactive oxygen species.

FIG. 18 depicts a graph showing X ray diffraction Pattern of Colloidal silver nanoparticles.

FIG. 19 depicts a graph showing X Ray diffraction pattern of pure curcumin. A solution of 5 mM methylene blue in D2O was irradiated overnight at 633 nm and ESI analyses were performed. FIG. 20 depicts ESI mass spectrum of methylene blue before and after overnight irradiation wherein; 20(a) is a spectrum before irradiation and 20(b) is a spectrum after irradiation.

FIG. 21 depicts graph showing nano silver mass spectrometry pattern.

FIG. 22 depicts Mass spectra Pattern of Curcumin; wherein 22(a) is mass spectra of curcumin 22(b) is mass spectra of hydroxylated curcumin and 22(c) is mass spectra of daughter ion of curcumin.

FIG. 23 depicts Absorption spectra of Nano gold; wherein 23(a) is a graph showing response of colloidal gold ion of 5 nm and 20 nm while 23(b) is a graph showing colloidal gold with low molecular complex.

FIG. 24 is a schematic overview of photo inactivation of multidrug resistant microbes by monomeric methylene blue conjugated gold nanoparticles.

FIG. 25 is a schematic overview of Methylene blue, curcumin and ion pairing nanoparticles effects on photodynamic therapy of Liposomal composition on cell line; wherein 25(a) shows effect in off-state and 25(b) shows effect in on-state.

FIG. 26 is a schematic representation of Tissue penetration depths of various wavelengths

FIG. 27 is a graph with the central image shows photo spectrometric data measured from penetration through a human hand in vivo. Based on the computer-derived trace on that part of the figure, the upper section illustrates relative penetration of selected wavelengths into the skin. Coupled with these, the lower section shows the absorption spectra of some biological chromophores, or targets, namely melanin, blood and water. Note the wavelength selectivity in these chromophores, and how that helps to determine the depth of penetration of different wavelengths into a living target as well as determining the target itself.

FIG. 28 is a schematic representation of general action mechanism of photodynamic applications on viruses. The PDI of viruses shares the general action mechanism of photodynamic applications: the irradiation of a dye with light and the subsequent generation of ROS which are the effective phototoxic agents damaging virus targets by reacting with viral nucleic acids, lipids and proteins.

FIG. 29 is a schematic representation of clinical photodynamic therapy for fighting respiratory tract infections: a promising tool against COVID-19.

### EXAMPLES AND DISCUSSION

### Example 1

### Process for Preparing a Liquid Formulation Comprising Methylene Blue.

In one embodiment described herein, the process for preparing the formulation comprises the following steps:
a. mixing glycerin and purified water to obtain diluted glycerin,
b. mixing methylene blue and purified water to obtain diluted methylene blue,
c. mixing the diluted glycerin from step (a) and diluted methylene blue from step (b) with the phospholipid,
d. hydrate/mix,
e. adding the nano or micronized gold or nano or micronized colloidal gold and/or the nano or micronized silver or nano or micronized colloidal silver to (d),
f. continue mixing at room temperature or in an ice bath,
g. adding PEG 400 to (g) and mixing, and
h. obtaining the pharmaceutical formulation.

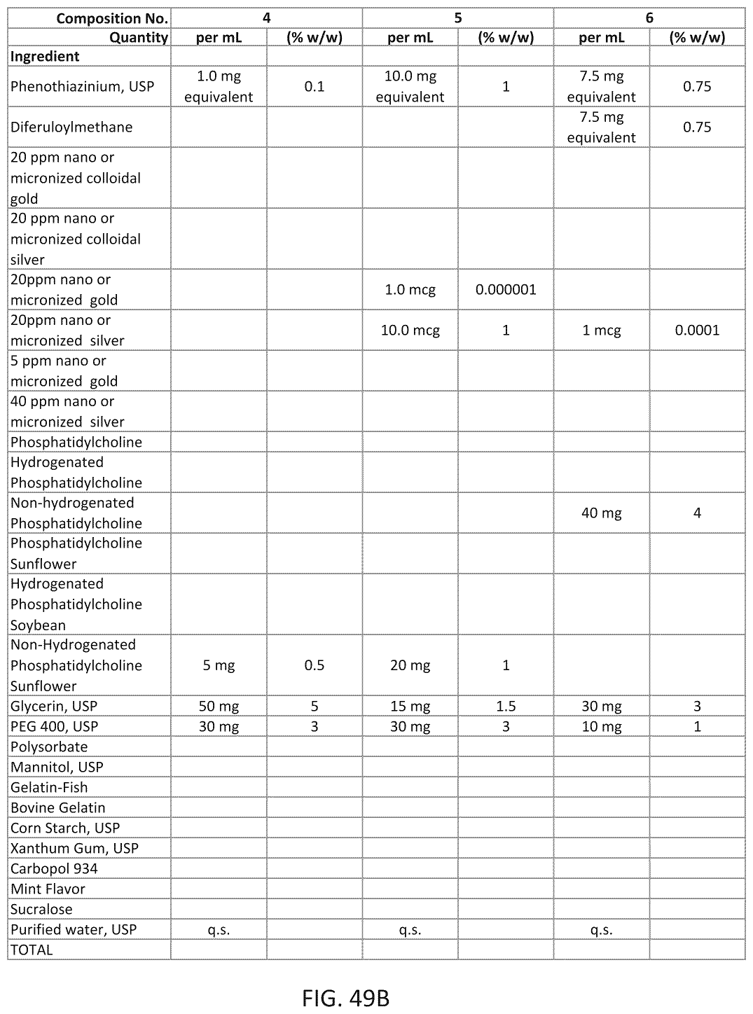

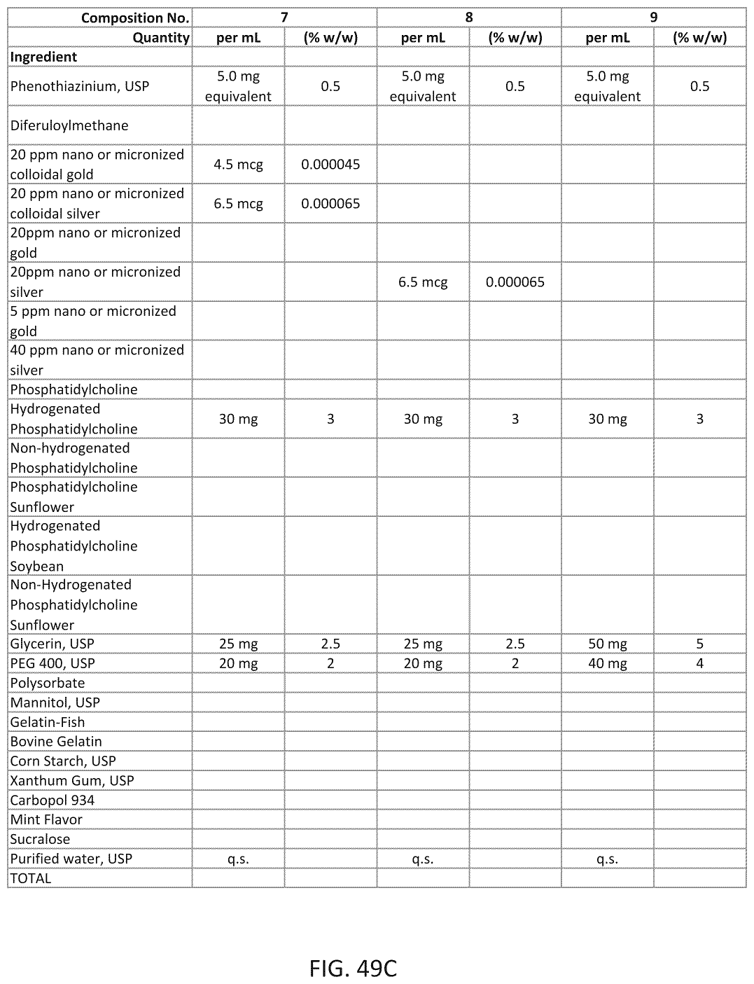

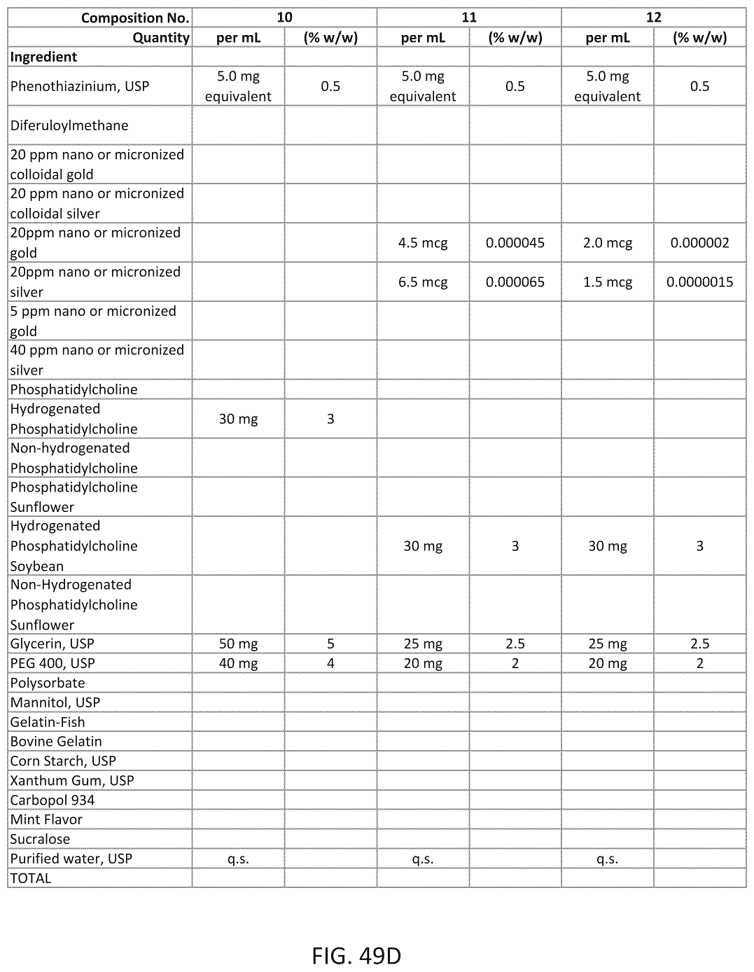

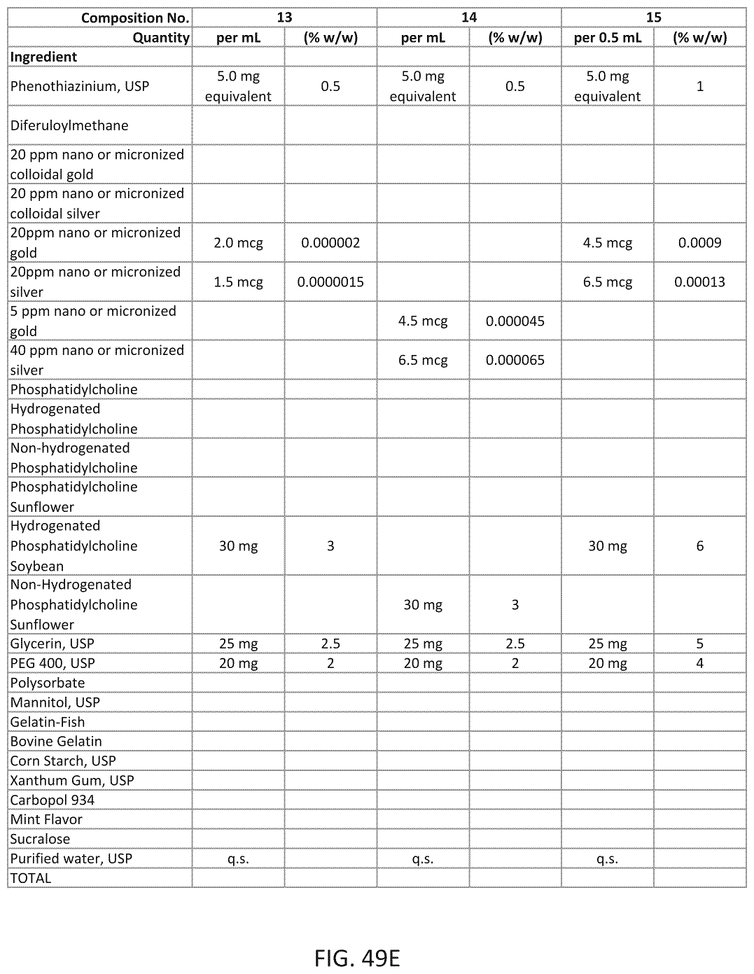

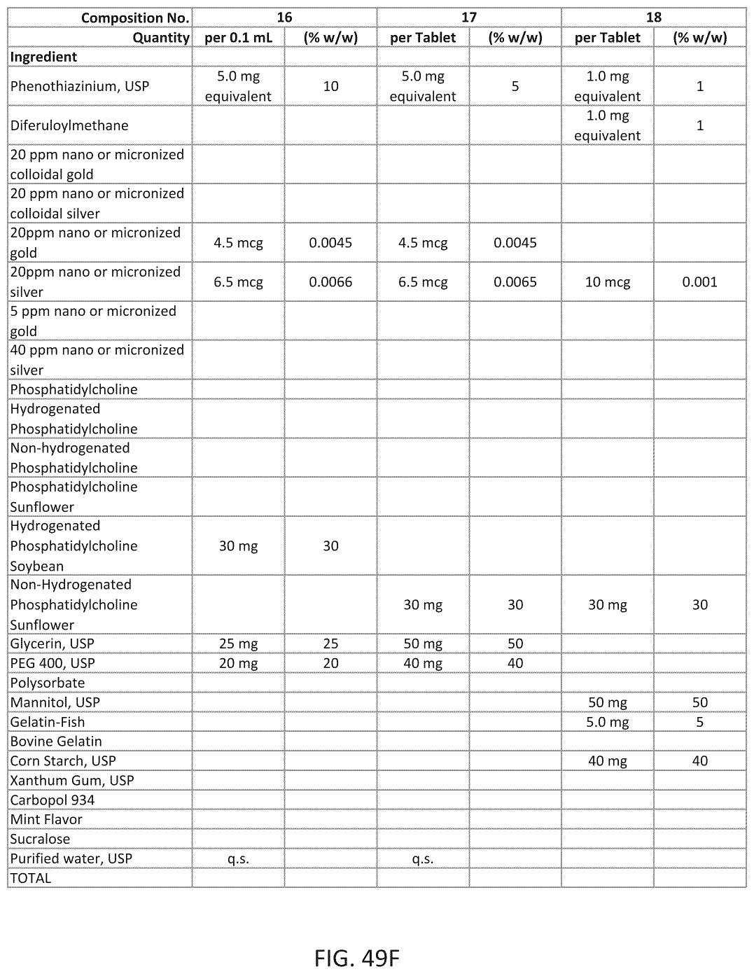

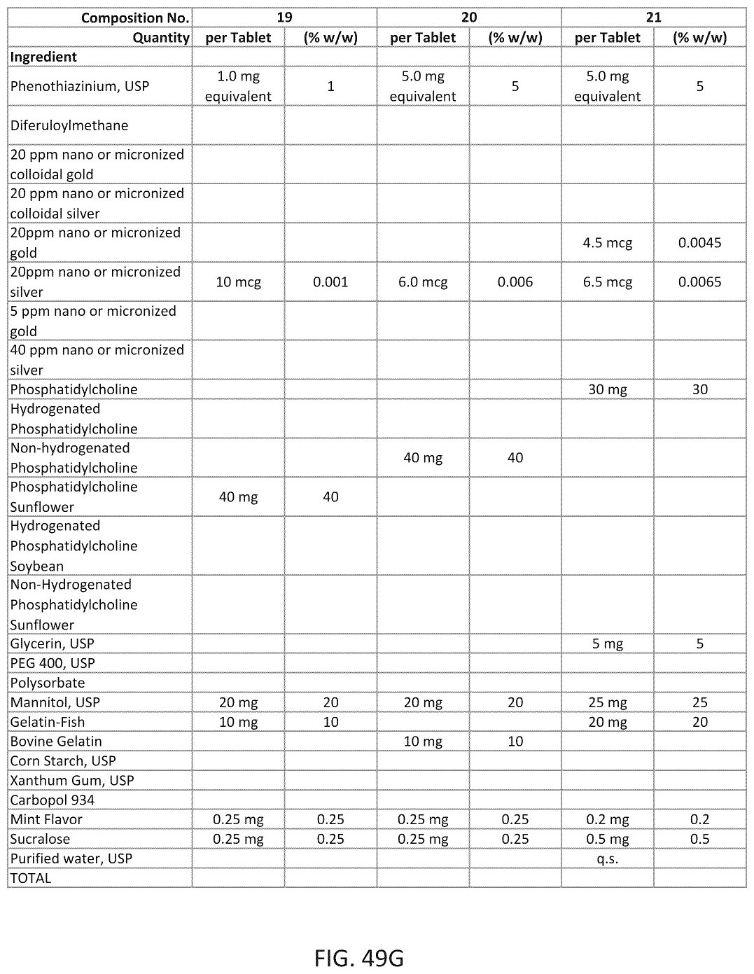

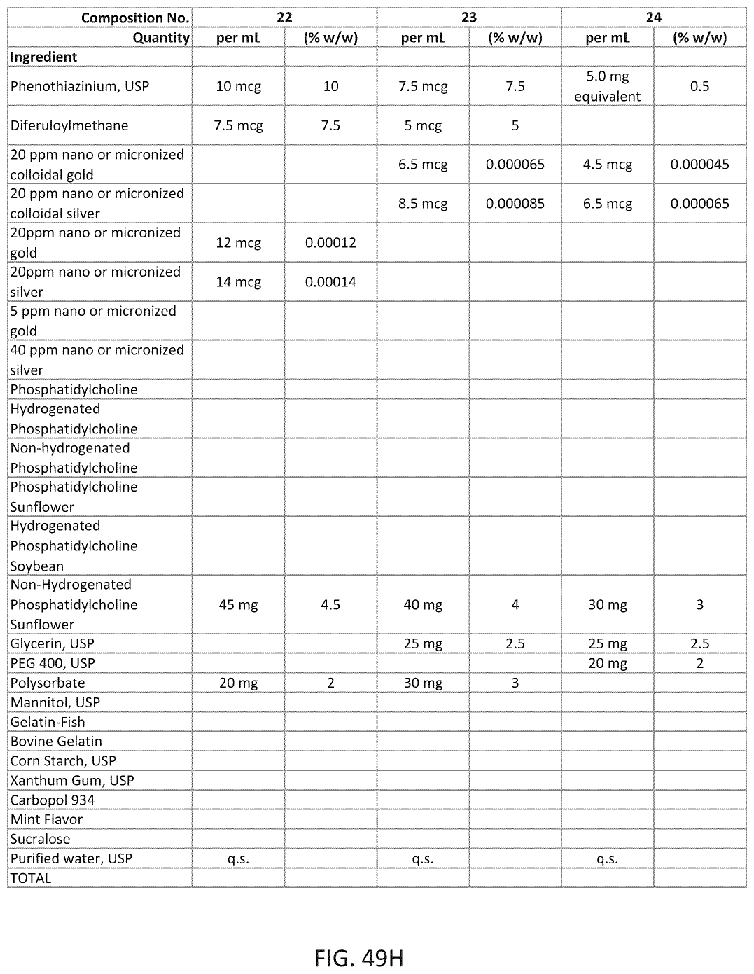

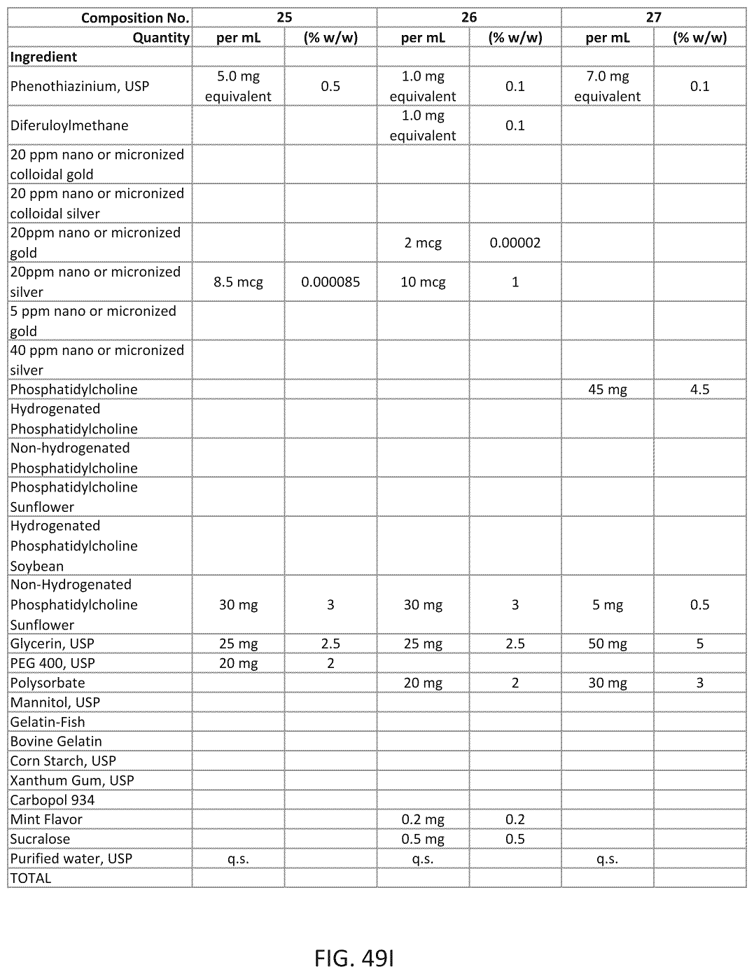

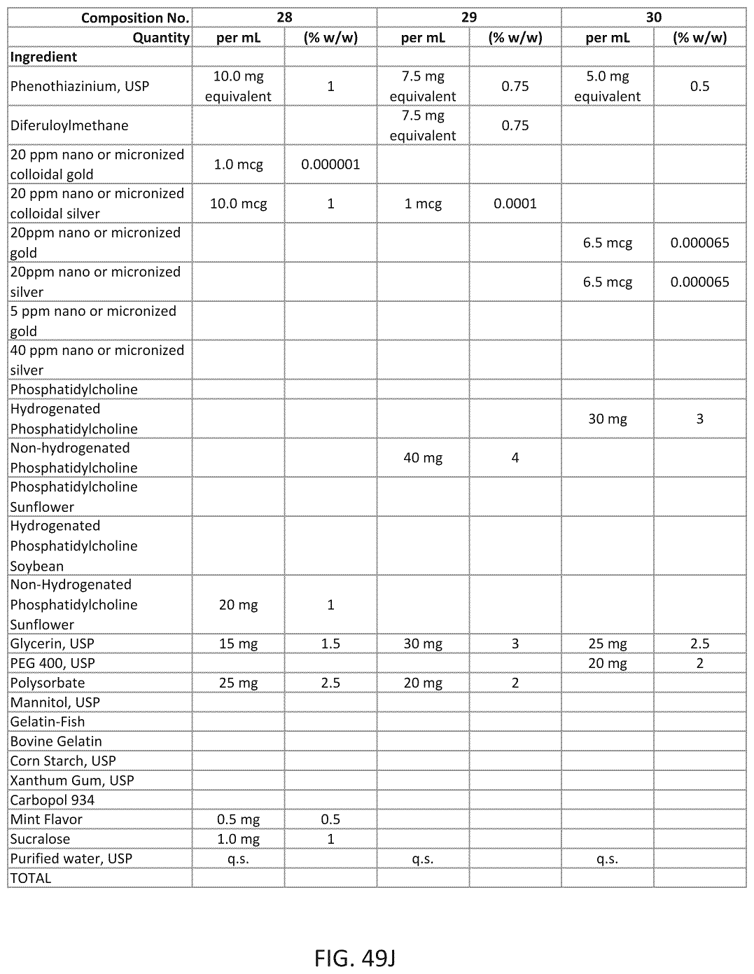

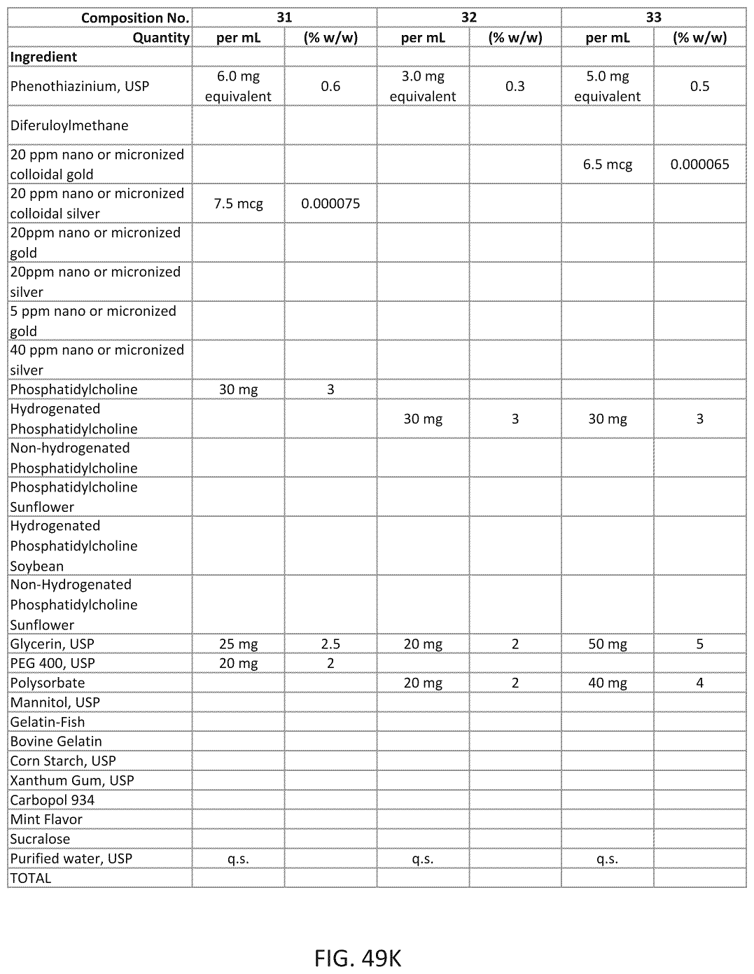

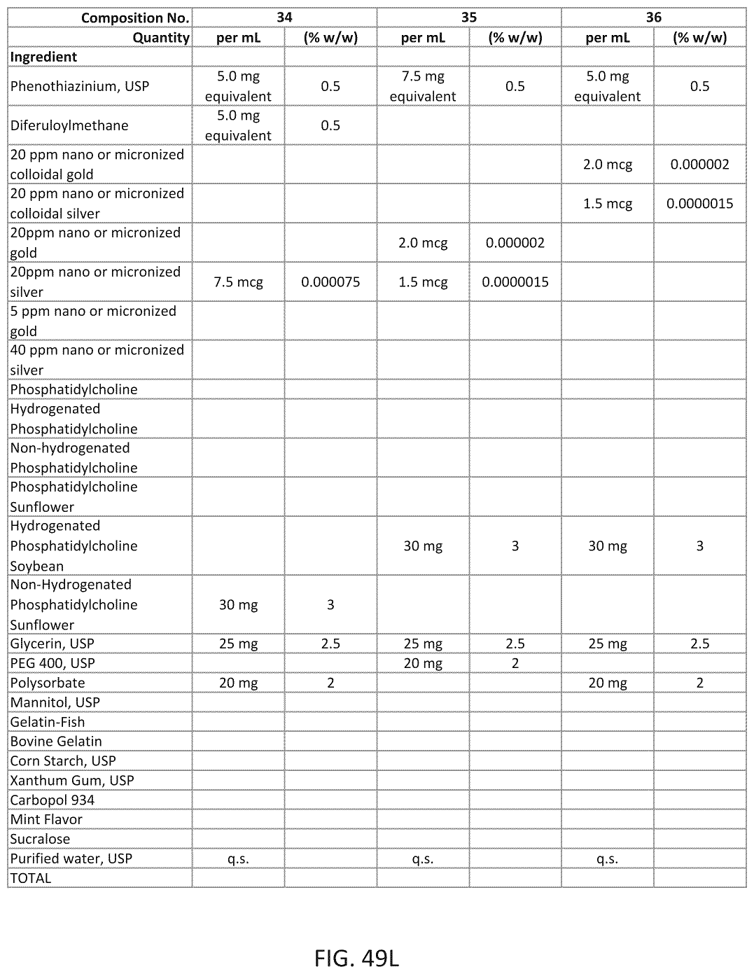

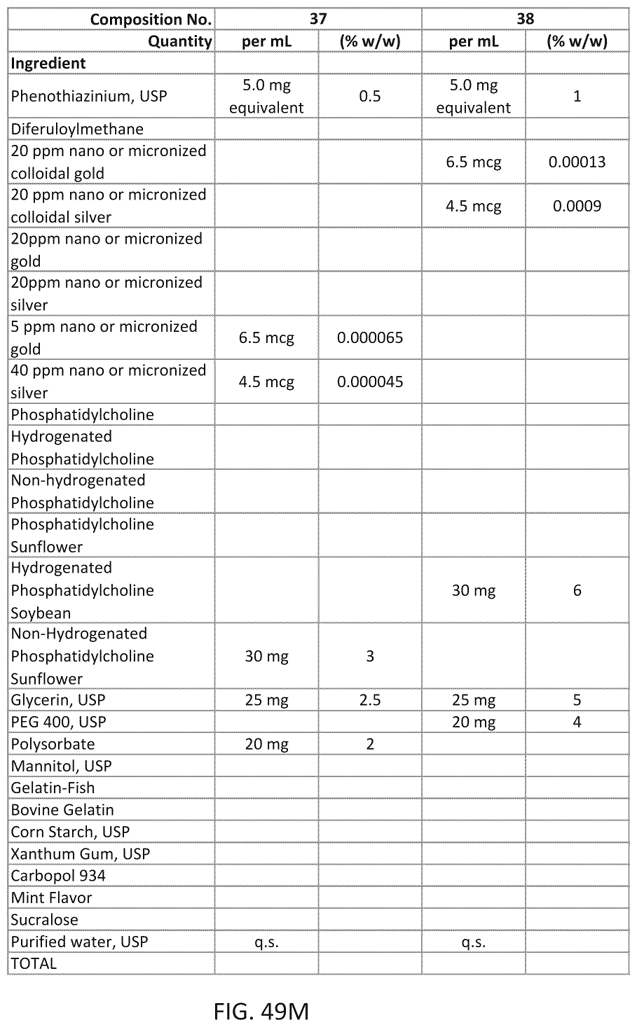

Examples of formulations that may be prepared by the above process can be seen in FIG. 49A to 49M.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A photosensitizer composition, said composition comprising:
(a) a therapeutically effective amount of phenothiazine compound;
(b) at least one phospholipid, or lysolipid or derivatives thereof;
(c) metal nanoparticles, wherein said metal is at least one selected from a group consisting of gold, silver, zinc, copper, iridium, platinum, silica, and palladium; and
(d) a suitable excipient, and a suitable solvent.

2. The composition as claimed in claim 1, wherein said phenothiazine compound is present in an amount in the range of 0.5 mg to 15.0 mg equivalent per ml of the total composition.

3. The composition as claimed in claim 1, wherein said phenothiazine compound is present in an amount in the range of 0.1% (w/w) to 15% (w/w), of the total composition.

4. The composition as claimed in claim 1, wherein said phenothiazinium compound is at least one compound, or analog or derivative thereof, selected from a group consisting of methylene blue, rose bengal, toluidine blue O, brilliant crystal blue, neutral red, pyronin Y, new methylene blue, dimethyl methylene blue, dimethyl methylene blue, Azure A, Azure B and Azure C.

5. The composition as claimed in claim 1, wherein said phospholipid is selected from the group consisting of lyso-phosphatidylcholine, hydrogenated phosphatidyl choline, non-hydrogenated phosphatidylcholine, hydrogenated sunflower phosphatidylcholine, non-hydrogenated sunflower phosphatidylcholine, hydrogenated soybean phosphatidylcholine, non-hydrogenated soybean phosphatidylcholine, egg yolk phosphatidylcholine, and hydrogenated egg yolk phosphatidylcholine.

6. An orally disintegrating tablet, comprising the composition claimed in claim 1.

7. A method for preparing the photosensitizer composition claimed in claim 1, said method comprising liquid-mixing said phenothiazine compound with said solvent to obtain diluted phenothiazine; adding said phospholipid, or lysolipid thereof, or derivatives thereof, and liquid mixing to obtain stabilized phenothiazine mixture; adding metal nano and/or micro particles; sonicating to achieve dispersion; adding one or more suitable excipients and mixing to obtain a stabilized composition.

8. The method as claimed in claim 6, wherein said mixing is performed to obtain a homogenous solution.

9. A method for treatment of microbial infections, said method comprising administering, to a subject in need thereof, a therapeutically effective amount of said photosensitizer composition claimed in claim 1.

10. The method as claimed in claim 1, said method comprising activating said photosensitizer composition by exposure to light energy of wavelength in the range of 600 nm to 700 nm.

11. A device comprising:
(a) a light source, wherein the light source comprises at least 1 LED light to the at least 10 LED lights;
(b) a battery, wherein the battery provides power to illuminate the at least 1 LED light to the at least 10 LED lights;
(c) one or more buttons, wherein the one or more buttons start or stop illumination of the at least 1 LED light to the at least 10 LED lights; and
(d) a cushion section, wherein the cushion section attaches to a predefined body location of a user.

12. The device according to claim 11, wherein the at least 1 LED light to the at least 10 LED lights emit a wavelength of about 600 nm to 750 nm.

13. The device according to claim 11, wherein the housing comprises a charging port.

14. The device according to of claim 11, wherein the wavelength of each of the at least 1 LED light to the at least 10 LED lights is the same or different.

15. The device according to claim 11, wherein the device further comprises a clear glass or plastic light pass area to allow the at least 1 LED light to the at least 10 LED lights to pass through the user's skin.
